(19) United States
(12) Patent Application Publication
(10) Pub. No.: US 2013/0078663 A1
(43) Pub. Date: Mar. 28, 2013

(54) Pore-Limit Electrophoresis (PLE) Microchannel Assays

(71) Applicants: Amy E. Herr, Oakland, CA (US); Xiofang Chen, Berkeley, CA (US)

(72) Inventors: Amy E. Herr, Oakland, CA (US); Xiofang Chen, Berkeley, CA (US)

(21) Appl. No.: 13/512,296

(22) PCT Filed: Dec. 1, 2010
(86) PCT No.: PCT/US10/58590

§ 371 Date: Aug. 2, 2012

Related U.S. Application Data
(60) Provisional application No. 61/266,060, filed on Dec. 2, 2009.

(51) Int. Cl. C12Q 1/28

(52) U.S. Cl. CPC C12Q 1/28
USPC 435/28; 422/502

(57) ABSTRACT

Pore-limit electrophoresis (PLE) microchannel assay methods are provided. Aspects of the methods include sequentially introducing samples at least suspected of containing first and second assay members into a PLE microchannel and then evaluating the microchannel for interaction between the first and second assay members. Aspects of the invention further include devices, systems and kits configured for practicing methods of invention. The methods, devices, systems and kits of the invention find use in a variety of different applications, including enzyme activity assays and immunoassays.

### CROSS-REFERENCE TO RELATED APPLICATIONS

Pursuant to 35 U.S.C. §119(e), this application claims priority to the filing date of U.S. Provisional Patent Application Ser. No. 61/266,060, filed Dec. 2, 2009, which application is incorporated herein by reference in its entirety.

### REFERENCE TO GOVERNMENT SUPPORT

This invention was made in part with government support under grant no. N66001-09-1-2118 awarded by DARPA. The government has certain rights in this invention.

### INTRODUCTION

Microfluidics refers to a set of technologies involving the flow of fluids through channels having at least one linear interior dimension, such as depth or radius, of less than 1 mm. It is possible to create microscopic equivalents of bench-top laboratory equipment such as beakers, pipettes, incubators, electrophoresis chambers, and analytical instruments within the channels of a microfluidic device. Since it is also possible to combine the functions of several pieces of equipment on a single microfluidic device, a single microfluidic device can perform a complete analysis that would ordinarily require the use of several pieces of laboratory equipment. A microfluidic device designed to carry out a complete chemical or biochemical analysis is commonly referred to as a micro-Total Analysis System (μ-TAS) or a “lab-on-a chip.”

A lab-on-a-chip type microfluidic device, which can simply be referred to as a “chip,” may be used as a replaceable component, like a cartridge or cassette, within an instrument. The chip and the instrument form a complete microfluidic system. The instrument can be designed to interface with microfluidic devices designed to perform different assays, giving the system broad functionality. For example, the commercially available Agilent 2100 Bioanalyzer system can be configured to interface with four different types of assays, namely, DNA (deoxyribonucleic acid), RNA (ribonucleic acid), protein and cell assays, by simply placing the appropriate type of chip into the instrument.

In certain microfluidic systems, all of the microfluidic channels are in the interior of the chip. The instrument can interface with the chip by performing a variety of different functions: supplying the driving forces that propel fluid through the channels in the chip, monitoring and controlling conditions (e.g., temperature) within the chip, collecting signals emanating from the chip, introducing fluids into and extracting fluids out of the chip, and possibly many others. The instruments may be computer controlled so that they can be programmed to interface with different types of chips and to interface with a particular chip in such a way as to carry out a desired analysis.

Microfluidic devices designed to carry out complex analyses may have complicated networks of intersecting channels with some of the channels being open to the outside of the microfluidic devices through one or more wells. Performing the desired assay on such chips may involve separately controlling the flows through certain channels and selectively directing flows from certain channels through channel intersections. Fluid flow through complex interconnected channel networks can be accomplished by either building microscopic pumps and valves into the chip or applying a combination of driving forces to the channels. The use of multiple electrical or pressure driving forces to control flow in a chip eliminates the need to fabricate valves and pumps on the chip itself, thus simplifying chip design and lowering chip cost.

Lab-on-a-chip type microfluidic devices offer a variety of inherent advantages over conventional laboratory processes such as reduced consumption of sample and reagents, ease of automation, large surface-to-volume ratios, and relatively fast reaction times. Thus, microfluidic devices have the potential to perform diagnostic assays more quickly, reproducibly, and at a lower cost than conventional devices. The advantages of applying microfluidic technology to diagnostic applications were recognized early on in development of microfluidics. For example, microfluidic systems exist in which the steps of sample preparation, PCR (polymerase chain reaction) amplification, and analyte detection are carried out on a single chip.

### SUMMARY

Pore-limit electrophoresis (PLE) microchannel assay methods are provided. Aspects of the methods include sequentially introducing samples at least suspected of containing first and second assay members into a PLE microchannel and then evaluating the microchannel for interaction between the first and second assay members. Aspects of the invention further include devices, systems and kits configured for practicing methods of invention. The methods, devices, systems and kits of the invention find use in a variety of different applications, including enzyme activity assays and immunoassays.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic of two-step PLENZ assay for enzyme molecular weight and activity in one separation channel. (a) A heterogeneous protein mixture is separated via PLE for 20 min. Here, the target enzyme CIP is present at low concentration compared to marker proteins. (b) During enzyme activity assay, post-PLE introduction of DiFMUP and subsequent “stopped-flow” conditions reveal axial location of CIP enzyme, with the resulting development of a blue product band yielding quantitative kinetic and sizing information. At bottom, conversion of weakly fluorescent DiFMUP substrate to strongly fluorescent DiFMU product by CIP under the Michaelis-Menten model.

FIG. 2. Quantitative PLENZ zymography by spectrally resolved protein (“green signal”) and product (“blue signal”) imaging. (a) Time-evolution of PLE protein sizing for a five component fluorescent protein ladder with 500 pM CIP enzyme (undetectable in green fluorescence profile, i=0.3 μA, ladder loading time=90 s). (b) Electrophoretic transport of 1000 μM (nominal) DiFMUP substrate across pseudo-immobilized proteins leads to production of blue fluorescent DiFMU, revealing the position of CIP. (c) The time derivative of the blue fluorescence profile (red) is compared to marker peaks (green) to yield CIP molecular weight. (d) Pseudo-color images of the final green fluorescence (top), and blue fluorescence 10 min into the assay phase place the product peak between the 150 and 80 kDa reference peaks (N.B. channel aspect ratios have been increased for clarity).

FIG. 3. Repeatable, linear protein sizing allows accurate inference of enzyme weight and local DiFMUP concentration. (a) Log-linear plot of protein molecular weight against migration distance at the end of the assay phase for 8 devices ( [symbol] , error bars are ±S.D.). Ladder results for the device of interest ( [symbol] ) illustrate how axial location of maximum enzyme activity reports the molecular weight of CIP (red point). (b) Position of maximum enzyme turnover also reports local DiFMUP concentration from a substrate distribution collected a priori (red point in (c)). For conceptual comparison, data from FIG. 2c is reproduced. (c) Axial DiFMUP concentration as a smoothed function of distance (blue lines) at 60 s intervals for the first 10 min of continuous loading (nominal [DiFMUP]=300 μM, i=0.3 μA). Raw and smoothed data for the sixth minute are shown in black, which otherwise marks the start of the stopped-flow portion of the assay phase.

FIG. 4. Enzyme activity scales linearly with amount of enzyme loaded during PLENZ. (a) Kinetics of DiFMU accumulation for 8 separate devices loaded with the indicated amounts of CIP (ladder loading times of 30-105 s, i=0.3 μA). Assays began at introduction of a saturating 1000 μM DiFMUP solution (i=0.3 μA). Linear product generation is observed after substrate introduction was halted at 6 min. (b) Reaction rate depends linearly on amount of CIP enzyme loaded (N=15 devices). Open and shaded squares denote devices run on separate days, colors correspond between (a) and (b).

FIG. 5. Saturating dependence of normalized enzyme activity on local substrate concentration yields kinetic parameters of CIP. (a) DiFMU accumulation after DiFMUP solutions of the indicated concentrations were loaded for 6 min into 8 separate devices (ladder loading time in PLE phase of 60 s at i=0.3 μA for all devices). (b) Normalized reaction rates against local DiFMUP concentration show a characteristic asymptotic relationship (N=15 devices, shaded and black circles denote devices run on separate days, colors correspond between (a) and (b)). Squares are data from FIG. 4 for ˜ saturating concentrations of DiFMUP (▪, N=8; □, N=7); error bars are ±S.D. Crosses (x) indicate microplate data. (c) Lineweaver-Burk plot of the PLENZ () and microplate (x) data from (b).

FIG. 6. (a) A segment of a PLENZ device at 4× magnification exposed under green (top) and orange-red (bottom) fluorescence conditions reveals the production of resorufin by HRP (nominal concentration of 100 pM) between the 80 kDa and 45 kDa marker peaks. Note that other molecular weight markers are also present in the channel but do not appear in the same field of view. (b) The resorufin signal is shown at several time points after measurement began under stopped-flow conditions, along with the initial green fluorescence signal.

FIG. 7. (a) UV photopatterning of a polyacrylamide membrane at the eventual 35% T end of the device is achieved in 60s using a slit in a black plastic mask. (b) Low percentage acrylamide solution is loaded into the opposite well, beginning the diffusion process. (c) 20 hours later, the pore size gradient is set via a 90s flood illumination under UV.

FIG. 8. (a) Average ladder separation resolution against the log of the pairwise molecular weight ratio for six representative PLENZ devices at the end of the separation phase (error bars are ±S.D.). (b) Blue fluorescence SNR at the end of the assay phase under DiFMUP-saturating conditions against amount of CIP loaded. (c) Amount of CIP enzyme added to the eight PLENZ devices in (b) against the ladder loading time at a constant current of 0.3 μA. These points correspond to the shaded squares in FIG. 4b.

FIG. 9. Principle of PLE-IA. Step 1: antibody immobilization. Step 2: sample injection. Protein specific to the antibody will be captured by the antibody. Step 3: detection. A fluorescently labeled reporter will be used to detect any target protein captured by the antibody.

FIG. 10. A schematic of the steps generally involved in PLE-IA. Red represents the antibody, green represents the protein.

FIG. 11. Migration of protein ladder in the PLE channel (left). A linear relationship between Log MW and migration distance was achieved (right). 1: TI, 2: BSA, 3: CRP, 4: IgG. Proteins were labeled with Alexa Fluor 488.

FIG. 12. PLE-IA of PSA. A: one green band was observed when PSA migrating through a channel without antibody. B: Migration of PSA in a channel preloaded with PSA antibody. Electrical field was removed for 5 minutes when PSA band overlapped with the antibody band to allow fully interaction of PSA with antibody. Unbound PSA migrated away from the complex after the electrical field was applied again. PSA and antibody were labeled green and red respectively.

FIG. 13. PLE-IA of S100. A: S100 migrated through a channel without antibody, only 1 green band was observed. B: two green bands were formed when S100 migrated through a channel preloaded with S100 antibody. PSA and antibody were labeled green and red respectively.

FIG. 14. Specificity of PLE-IA. a: PSA and S100 migrating through a channel without antibody. b: PSA and S100 migrating through a channel preloaded with PSA antibody. c: PSA and S100 migrating through a channel with S100 antibody. PSA was labeled red. S100 was labeled green. Antibody was not labeled.

FIG. 15. PLE-IA of FST. A: Separation of the free FST from FST-antibody complex in a PLE-IA channel. B: Position of ladder proteins ran parallel with FST in a distinct channel without antibody.

FIG. 16. Dose response curve of FST PLE-IA (panel A). FST was injected for 30 seconds. A linear relationship between FL signal of the complex and initial FST concentration was achieved in the range from 5 nM to 1333 nM. TI was used as an internal control. Panel B and C show the complex, free FST and TI bands in the channel when initial FST concentration was 21 and 1333 nM.

FIG. 17. Continuous injection of ladder protein. Protein ladder (TI, BSA, CRP, IgG) was injected into the PLE channel continuously. The front of each protein can be observed (left and right upper). Migration distance of the front is proportional to log molecular weight (right lower).

FIG. 18. Continuous formation of complex during injection. FST was injected into a PLE channel preloaded with FST antibody for 40 minutes. FL signal from the complex were plotted against time in the right panel.

FIG. 19. Detection and sizing of FST from protein mixture. A mixture of TI, FST and BSA was injected into PLE channels with (lower) or without (upper) antibody. Fronts of each protein can be observed at the end of injection (upper, left). Protein bands were resolved after 10 minutes wash with clear buffer (upper, right). The free FST band was reduced significantly in the channel with antibody while a complex band which overlapped with the antibody band (red curve) was formed (lower).

FIG. 20. Dose response curve of FST PLE-IA when FST was injected into the channel preloaded with antibody continuously for 40 minutes.

### DETAILED DESCRIPTION

Pore-limit electrophoresis (PLE) microchannel assay methods are provided. Aspects of the methods include sequentially introducing samples at least suspected of containing first and second assay members into a PLE microchannel and then evaluating the microchannel for interaction between the first and second assay members. Aspects of the invention further include devices, systems and kits configured for practicing methods of invention. The methods, devices, systems and kits of the invention find use in a variety of different applications, including enzyme activity assays and immunoassays.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

As summarized above, methods of performing pore-limit electrophoresis (PLE) microchannel assay methods are provided are provided. As the assays of the invention are PLE microchannel assays, they are assays which include a step or procedure that is carried out in a PLE microchannel. Accordingly, some portion of the assays of embodiments of the invention is performed in a PLE microchannel. PLE microchannels are microchannels, e.g., as described in greater detail below, which include a separation medium having a pore size gradient along a length of the microchannel, such that the pore size changes, e.g., increases or decreases, as one moves from one portion of the microchannel to the other. In some instances, the gradient is linear, such that the pore size changes in a linear fashion along the length of the microchannel. The length of a given PLE microchannel (i.e., the length of a given microchannel that is occupied by a separation medium having a pore size gradient) may vary. In some instances, the length is 2 mm or longer, such as 5 mm or longer, e.g., 7.5 mm or longer. In certain embodiments, the length of a given PLE microchannel may range from 2 to 15 mm, such as 3 to 10 mm, including 5 to 7 mm. In certain embodiments, the microfluidic channels have a width ranging from 1 μm to 500 μm, such as from 5 μm to 300 μm, including from 10 μm to 200 μm, for example from 50 μm to 150 μm. In some instances, the microfluidic channels have a width of 100 μm. In certain embodiments, the microfluidic channels have a depth ranging from 1 μm to 200 μm, such as from 5 μm to 100 μm, including from 10 μm to 50 μm. In some cases, the microfluidic channels have a depth of 25 μm.

As described in the art, the porosity gradient may be described in terms of the concentration of at least the monomer (% T) that is present in the microchannel prior to polymerization. In some instances, the concentration of monomer in the microchannel prior to polymerization may range from 2% T to 50% T, such as 5% to 45% T, including 10% to 40% T along the length of the microchannel (such that the monomer concentration (% T) increases from one end of the microchannel to the other, where the increase may be in a linear fashion). The crosslinker concentration (% C) may be constant or vary along the length of the microchannel, ranging from 1 to 15%, such as 2 to 13%. In some instances, the PLE microchannel may include a small pore membrane (e.g., fabricated from polymerization of a high concentration monomer precursor (such as one ranging from 30 to 50% T, e.g., 35 to 45% T) located at one end of the microchannel, which membrane is employed during fabrication of the microchannel, such as described below. Conditions in the PLE microchannel may be denaturing or non-denaturing, as desired.

Any convenient separation medium may be employed in the PLE microchannel. In certain embodiments, the PLE microchannel includes a polymer, such as a polymeric gel. The polymeric gel may be a gel suitable for gel electrophoresis. The polymeric gel may include, but is not limited to, a polyacrylamide gel, an agarose gel, and the like. Accordingly, the gel may be fabricated from suitable polymers, such as acrylate polymers, alkylacrylate polymers, alkyl alkylacrylate polymers, copolymers thereof, and the like. Any convenient cross-linker may be employed, where cross-linkers of interest include, but are not limited to: bis-acrylamide, etc.

As summarized above, aspects of the methods include sequentially introducing samples at least suspected of containing first and second assay members into a PLE microchannel and then evaluating the microchannel for interaction between the first and second assay members. The phrase “at least suspected” is used to describe embodiments where it is known that a given assay member, e.g., the first and/or second assay member, is present in the sample as well as embodiments where it is unknown prior to performing the method whether or not a given assay member is present in the sample. Examples of embodiments where the presence of the first assay member in the sample may just be suspected but not known for sure include assays where the first assay member is an analyte, e.g., as exemplified by the PLENZ assay embodiments described below in which the assay is one which provides information about whether a given enzyme is present in a sample. Examples of embodiments where the presence of the second assay member in a sample may just be suspected but not known for sure include assays where the first assay member is an analyte, e.g., as exemplified by the PLE-IA assay embodiments described below in which the assay is one which provides information about whether a given analyte is present in a second sample and the first assay member of the first sample is a specific binding pair member, e.g., antibody or binding fragment thereof, that specifically binds to the analyte.

As indicated above, the first and second assay members may vary widely depending upon a given assay. The first and second assay members may be analytes, reagent members (such as enzymes substrates, labeled reporter molecules, etc.) which interact with other in some manner during a given assay. As used herein, the term reagent refers to “a substance or compound that is added to a system in order to bring about a chemical reaction or is added to see if a reaction occurs.” IUPAC Gold Book internet edition: (1996). The interaction that occurs between the assay members may vary depending on a particular first and second assay member pair, where interactions of interest include binding interactions, substrate-to-product conversion applications, etc.

As the methods include sequentially introducing samples of the first and second assay members into a PLE microchannel, in practicing the methods a first sample at least suspected of including a first assay member is introduced into a pore limit electrophoresis (PLE) microchannel. Next, a second sample at least suspected of including a second assay member is introduced into the PLE microchannel. Accordingly, the second sample at least suspected of including a second assay member is introduced into the PLE microchannel after the first sample at least suspected of including the first assay member.

Samples may be introduced into the PLE microchannel using any convenient protocol. Protocols of interest include but are not limited to positioning a quantity of sample at a first end of the PLE microchannel and then applying an electric field to the microchannel in a manner sufficient draw the sample into the microchannel. Examples of parameters (e.g., ranges of applied electric fields, etc.) for these steps are further described below. In some embodiments, the first sample is introduced into the microchannel in a manner sufficient for the first assay reagent to be pseudo-immobilized in the PLE microchannel if it is present in the sample. Thus, in these instances the electric field is applied in a way (e.g., in terms of strength and/or duration) such that the first assay member, if present, proceeds down the microchannel to a position where its movement substantially stops, e.g., such that further movement is substantially if not completely undetectable, at least during the duration of the assay being performed.

Following sequential introduction of first and second samples into the PLE microchannel, aspects of the invention include evaluating the PLE microchannel for interaction between the first and second assay reagent. By evaluating is meant assessing or determining whether an interaction between first and second assay members has occurred. The specific nature of the evaluating may vary depending on the particular assay being performed, where evaluating may include detecting the presence of label, a detectable product, etc., in the microchannel. Depending on the particular nature of the species to be detected, evaluating may include exciting a least a portion of the microchannel with one or more wavelengths of light, detected a fluorescent signal, etc.

In some instances, the signal that is obtained during the evaluation is a “real-time” signal. Accordingly, aspects of the methods may also include obtaining a real-time signal from the PLE microchannel. By “real-time” is meant that a signal is observed as it is being produced or immediately thereafter. Accordingly, certain embodiments include observing the evolution in real time of the signal associated with the occurrence of the interaction of interest (e.g., the binding of the analyte of interest to a pseudo-immobilized antibody, the enzyme mediated production of a detectable product from a substrate). The real-time signal may include two or more data points obtained over a given period of time, where in certain embodiments the signal obtained is a continuous set of data points (e.g., in the form of a trace) obtained continuously over a given period of time of interest. The time period of interest may vary, ranging in some instances from 0.1 min to 60 min, such as 0.5 min to 30 min, including 2 min to 15 min. The number of data points in the signal may also vary, where in some instances, the number of data points is sufficient to provide a continuous stretch of data over the time course of the real-time signal.

The first and second samples employed in methods of the invention may vary widely. Samples of interest include aqueous liquids that may be simple or complex samples. Simple samples are samples that include an assay member (e.g., a reagent), and may or may not include one or more molecular entities that are not the assay member, where the number of these non-interest molecular entities may be low, e.g., 10 or less, 5 or less, etc. Simple samples may include initial biological or other samples that have been processed in some manner, e.g., to remove potentially interfering molecular entities from the sample. By “complex sample” is meant a sample that may or may not have the assay member (e.g., analyte) of interest, but also includes many different proteins and other molecules that are not of interest. In some instances, the complex sample assayed in the subject methods is one that includes 10 or more, such as 20 or more, including 100 or more, e.g., 10³ or more, 10⁴ or more (such as 15,000; 20,000 or 25,000 or more) distinct (i.e., different) molecular entities, that differ from each other in terms of molecular structure or physical properties (e.g., molecular weight, size, charge, isoelectric point, etc.).

In certain embodiments, at least one of the first and second samples employed in methods of the invention is a biological sample, such as, but not limited to, urine, blood, serum, plasma, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue (e.g., samples obtained from laser capture microdissection (LCM)), and the like. The sample can be a biological sample or can be extracted from a biological sample derived from humans, animals, plants, fungi, yeast, bacteria, tissue cultures, viral cultures, or combinations thereof using conventional methods for the successful extraction of DNA, RNA, proteins and peptides. In certain embodiments, the sample is a fluid sample, such as a solution of analytes in a fluid. The fluid may be an aqueous fluid, such as, but not limited to water, a buffer, and the like.

In some instances, assay methods of invention may be viewed as analyte detection methods, where the first or second assay member is an analyte of interest and the other assay member is a reagent. Accordingly, aspects of the invention include determining whether an analyte is present in a sample, e.g., determining the presence or absence of one or more analytes in a sample. In certain embodiments of the methods, the presence of one or more analytes in the sample may be determined qualitatively or quantitatively. Qualitative determination includes determinations in which a simple yes/no result with respect to the presence of an analyte in the sample is provided to a user. Quantitative determination includes both semi-quantitative determinations in which a rough scale result, e.g., low, medium, high, is provided to a user regarding the amount of analyte in the sample and fine scale results in which an exact measurement of the concentration of the analyte is provided to the user. As described above, the samples that may be assayed in the subject methods may include one or more analytes of interest. Examples of detectable analytes include, but are not limited to: nucleic acids, e.g., double or single-stranded DNA, double or single-stranded RNA, DNA-RNA hybrids, DNA aptamers, RNA aptamers, etc.; proteins and peptides, with or without modifications, e.g., enzymes, antibodies, diabodies, Fab fragments, DNA or RNA binding proteins, phosphorylated proteins (phosphoproteomics), peptide aptamers, epitopes, and the like; small molecules such as inhibitors, activators, ligands, etc.; oligo or polysaccharides; mixtures thereof; and the like.

In some embodiments, the analyte of interest can be identified so that the presence of the analyte of interest can then be detected. Analytes may be identified by any of the methods described herein. For example, the analyte may include a detectable label. Detectable labels include, but are not limited to, fluorescent labels, colorimetric labels, chemiluminescent labels, enzyme-linked reagents, multicolor reagents, avidin-streptavidin associated detection reagents, non-visible detectable labels (e.g., radiolabels, gold particles, magnetic labels, electrical readouts, density signals, etc.), and the like. In certain embodiments, the detectable label is a fluorescent label. Fluorescent labels are labeling moieties that are detectable by a fluorescence detector. For example, binding of a fluorescent label to an analyte of interest may allow the analyte of interest to be detected by a fluorescence detector. Examples of fluorescent labels include, but are not limited to, fluorescent molecules that fluoresce upon contact with a reagent, fluorescent molecules that fluoresce when irradiated with electromagnetic radiation (e.g., UV, visible light, x-rays, etc.), and the like.

Suitable fluorescent molecules (fluorophores) include, but are not limited to, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like. Labels, e.g., as described above, may also be incorporated into suitable reporter molecules, such as specific binders to the analyte.

In certain embodiments, the methods further include introducing one or more reporter agents into the microchannel. The reporter agents may include a specific binding domain and a label domain. The specific binding domain specifically binds a target of interest, e.g., the second assay member, etc. The specific binding domain may be any convenient binding domain. A variety of different types of specific binding agents may be employed as the capture ligands. Specific binding agents of interest include antibody binding agents, proteins, peptides, haptens, nucleic acids, etc. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

In certain embodiments, the method includes concentrating, diluting, or buffer exchanging the sample prior to directing the sample through the separation medium. Concentrating the sample may include contacting the sample with a concentration medium prior to contacting the sample with the separation medium. As described above, the concentration medium may include a small pore size polymeric gel, a membrane (e.g., a size exclusion membrane), combinations thereof, and the like. Concentrating the sample prior to contacting the sample with the separation medium may facilitate an increase in the resolution between the bands of analytes in the separated sample because each separated band of analyte may disperse less as the sample traverses through the separation medium. Diluting the sample may include contacting the sample with additional buffer prior to contacting the sample with the separation medium. Buffer exchanging the sample may include contacting the sample with a buffer exchange medium prior to contacting the sample with the separation medium. The buffer exchange medium may include a buffer different from the sample buffer. The buffer exchange medium may include, but is not limited to, a molecular sieve, a porous resin, and the like.

In certain embodiments, the method includes transferring moieties that are not bound by the binding members in the binding medium away from the binding medium. The unbound moieties may be directed to a transfer flow path that is in fluid communication with the labeling flow path of the binding medium. In some cases, the method includes transferring the unbound moieties to a waste reservoir. In other cases, the method includes directing the unbound moieties downstream from the binding medium for secondary analysis with a secondary analysis device such as, but is not limited to, a UV spectrometer, and IR spectrometer, a mass spectrometer, an HPLC, an affinity assay device, and the like.

Embodiments of the method may also include releasing one or more assay members from the PLE microchannel. The releasing may include contacting the first and second assay members with a releasing agent. The releasing agent may be configured to disrupt the binding interaction between the first and second assay members. In some cases, the releasing agent is a reagent, buffer, or the like, that disrupts the binding interaction between an analyte and a binding member therefore causing the binding member to release the analyte. After releasing the analyte from the binding member, the method may include transferring the analyte away from the PLE microchannel. For example, the method may include directing the released analyte downstream from the PLE microchannel for secondary analysis with a secondary analysis device such as, but is not limited to, a UV spectrometer, and IR spectrometer, a mass spectrometer, an HPLC, an affinity assay device, and the like.

In some embodiments, the methods include the uniplex analysis of an analyte in a sample. By “uniplex analysis” is meant that a sample is analyzed to detect the presence of one analyte in the sample. For example, a sample may include a mixture of an analyte of interest and other molecular entities that are not of interest. In some cases, the methods include the uniplex analysis of the sample to determine the presence of the analyte of interest in the sample mixture.

Certain embodiments include the multiplex analysis of two or more analytes in a sample. By “multiplex analysis” is meant that the presence two or more distinct analytes, in which the two or more analytes are different from each other, is determined. For example, analytes may include detectable differences in their molecular weight, size, charge (e.g., mass to charge ratio), isoelectric point, and the like. In some instances, the number of analytes is greater than 2, such as 4 or more, 6 or more, 8 or more, etc., up to 20 or more, e.g., 50 or more, including 100 or more, distinct analytes. In certain embodiments, the methods include the multiplex analysis of 2 to 100 distinct analytes, such as 4 to 50 distinct analytes, including 4 to 20 distinct analytes.

In certain embodiments, the method is an automated method. As such, the method may include a minimum of user interaction with the microfluidic devices and systems after introducing the sample into the microfluidic device. For example, the steps of directing the sample through the separation medium to produce a separated sample and transferring the separated sample to the binding medium may be performed by the microfluidic device and system, such that the user need not manually perform these steps. In some cases, the automated method may facilitate a reduction in the total assay time. For example, embodiments of the method, including the separation and detection of analytes in a sample, may be performed in 30 min or less, such as 20 min or less, including 15 min or less, or 10 min or less, or 5 min or less, or 2 min or less, or 1 min or less.

In certain instances, in addition to the first and second samples, a molecular weight ladder is introduced into the PLE microchannel. Any convenient ladder may be introduced into the channel, where the constituent members of the ladder may or may not be pre-labeled, as desired. The term “ladder” is used in its conventional sense in the art to refer to an aqueous volume of liquid which includes a collection of distinct molecular entities, such as proteins, of known weight, where the concentration of each constituent member in the volume of liquid may also be known.

### PLENZ

In some instances, the methods are methods of determining whether an enzyme is present in a sample. Aspects of these methods include separating a sample in a pore limit electrophoresis (PLE) microchannel, e.g., by applying an electric field to the PLE microchannel in a manner sufficient to separate the constituents of the sample. Following sample separation, a substrate for the enzyme of interest is introduced into the PLE microchannel. After the substrate is introduced and a sufficient amount of time has passed for product to be produced by the enzyme from the substrate if the enzyme is present in the sample (and therefore present in the PLE microchannel), the PLE microchannel is evaluated for enzyme mediated production of product from the substrate to determine whether the enzyme is present in the sample. Accordingly, the presence of enzyme produced product in the microchannel is detected. The product may be directly detectable or, if not, then detected using a convenient reporter molecule, such as a dye that specifically binds to the product. In these embodiments, the first assay member is the enzyme and the second assay member is the substrate that is converted to product by the enzyme.

In some instances, the methods include determining a parameter of the enzyme that is present in the sample. A variety of different parameters may be determined, where parameters of interest include molecular weight and kinetic parameters. For example, where a ladder is run the microchannel, molecular weight of the enzyme may readily be determined by comparing the location of product to the ladder and then determining the molecular weight of the enzyme that produced the product based on the location of the product relative to the ladder. In those instances where a real-time signal is obtained, kinetic parameters may be determined. Kinetic parameters of interest include, but are not limited to: k_{cat}, Kₘ, etc.

Methods according to embodiments of the invention may have high detection sensitivity. In some instances, the detection sensitivity of the methods may be 50 zmol or less, such as 25 zmol or less, including 15 zmol or less, e.g., 10 zmol or less, including 5 zmol or less. As mentioned above, methods may include qualitatively or quantitatively determining whether the enzyme is present in the sample, the latter providing information on the amount of the enzyme in the sample that is assayed.

Methods according to embodiments of the invention may be performed in rapid time, e.g., in 120 minutes or less, such as 90 minutes or less, including 60 minutes or less, e.g., 30 minutes or less.

As reviewed above, methods of invention may be uniplex in that a single enzyme of interest is detected or multiplex, in which two or more different enzymes of interest are detected, and in some instances characterized, e.g., in terms of determination of molecular weight, kinetic parameter, amount in the sample, etc.

### PLE-IA

As reviewed above, embodiments of the invention are directed to methods of determining whether an analyte is present in a sample. In certain of these embodiments, the methods include pseudo-immobilizing a specific binding pair member, e.g., antibody or binding fragment thereof, that specifically binds to the analyte of interest in a pore limit electrophoresis (PLE) microchannel. Following pseudo-immobilization of the specific binding member in the PLE microchannel, the methods include introducing a sample to be assayed for the analyte into the PLE microchannel. Following this step, the PLE microchannel is evaluated for the presence of binding member-analyte (e.g., antibody-analyte) complex to determine whether the analyte is present in the sample. To evaluate the complex, the analyte may itself be labeled or a labeled reporter molecule that specifically binds to the complex may be employed. In these embodiments, the first assay member is the binding member and the second assay member is the analyte.

Aspects of these methods may include determining a parameter of the analyte when the analyte is determined to be present in the sample. As reviewed above, in some instances the parameter is molecular weight, which may be determined using protocols analogous to those summarized above. In those instances where a real-time signal is obtained, binding kinetic parameters may be determined. By “binding kinetic parameter” is meant a measurable binding kinetic factor that at least partially defines a given molecular interaction and can be employed to define its behavior. Binding kinetic parameters of interest include, but are not limited to, association constants (i.e., kₐ, kₒₙ), dissociation constants (i.e., k_{d}, k_{off}), diffusion constant (i.e., k_{M}), activation energy (i.e., E_{A}), transport parameters such as diffusivity, etc.

Methods according to embodiments of the invention may have high detection sensitivity. In some instances, the detection sensitivity of the methods may be 50 zmol or less, such as 25 zmol or less, including 15 zmol or less, e.g., 10 zmol or less, including 5 zmol or less. As mentioned above, methods may include qualitatively or quantitatively determining whether the analyte is present in the sample, the latter providing information on the amount of the analyte in the sample that is assayed.

Methods according to embodiments of the invention may be performed in rapid time, e.g., in 120 minutes or less, such as 90 minutes or less, including 60 minutes or less, e.g., 30 minutes or less.

As reviewed above, methods of invention may be uniplex in that a single analyte f interest is detected or multiplex, in which two or more different analytes of interest are detected, and in some instances characterized, e.g., in terms of determination of molecular weight, kinetic parameter, amount in the sample, etc.

### Systems

Aspects of certain embodiments include a system configured to perform methods of the invention, e.g., as described above. In some instances, the system includes a microfluidic device as described herein. The system may also include a detector. In some cases, the detector is a detector configured to detect a detectable label. As described above, the detectable label may be a fluorescent label. For example, the fluorescent label can be contacted with electromagnetic radiation (e.g., visible, UV, x-ray, etc.), which excites the fluorescent label and causes the fluorescent label to emit detectable electromagnetic radiation (e.g., visible light, etc.). The emitted electromagnetic radiation may be detected with an appropriate detector to determine the presence of the analyte bound to the binding member.

In some instances, the detector may be configured to detect emissions from a fluorescent label, as described above. In certain cases, the detector includes a photomultiplier tube (PMT), a charge-coupled device (CCD), an intensified charge-coupled device (ICCD), a complementary metal-oxide-semiconductor (CMOS) sensor, a visual colorimetric readout, a photodiode, and the like. This detector may be part of a reader configured to evaluate the PLE microchannel to obtain a signal.

Systems of the present disclosure may include various other components as desired. For example, the systems may include fluid handling components, such as microfluidic fluid handling components. The fluid handling components may be configured to direct one or more fluids through the microfluidic device. In some instances, the fluid handling components are configured to direct fluids, such as, but not limited to, sample solutions, buffers (e.g., release buffers, wash buffers, electrophoresis buffers, etc.), and the like. In certain embodiments, the microfluidic fluid handling components are configured to deliver a fluid to the separation medium of the microfluidic device, such that the fluid contacts the separation medium. The fluid handling components may include microfluidic pumps. In some cases, the microfluidic pumps are configured for pressure-driven microfluidic handling and routing of fluids through the microfluidic devices and systems disclosed herein. In certain instances, the microfluidic fluid handling components are configured to deliver small volumes of fluid, such as 1 mL or less, such as 500 μL or less, including 100 μL or less, for example 50 μL or less, or 25 μL or less, or 10 μL or less, or 5 μL or less, or 1 μL or less.

In certain embodiments, the systems include one or more electric field generators. An electric field generator may be configured to apply an electric field to various regions of the microfluidic device. The system may be configured to apply an electric field such that the sample is electrokinetically transported through the microfluidic device. For example, the electric field generator may be configured to apply an electric field to the PLE microchannel. In some cases, the applied electric field may be aligned with the directional axis of the separation flow path of the separation medium. As such, the applied electric field may be configured to electrokinetically transport the assay members and moieties in a sample through the PLE microchannel. In some instances, the electric field generators are configured to apply an electric field with a strength ranging from 10 V/cm to 1000 V/cm, such as from 100 V/cm to 800 V/cm, including from 200 V/cm to 600 V/cm.

In certain embodiments, the electric field generators include voltage shaping components. In some cases, the voltage shaping components are configured to control the strength of the applied electric field, such that the applied electric field strength is substantially uniform across the PLE microchannel. The voltage shaping components may facilitate an increase in the resolution of the assay members in the sample. For instance, the voltage shaping components may facilitate a reduction in non-uniform movement of the sample through the separation medium. In addition, the voltage shaping components may facilitate a minimization in the dispersion of the bands of analytes as the analytes traverses the separation medium.

In certain embodiments, the subject system is a biochip (e.g., a biosensor chip). By “biochip” or “biosensor chip” is meant a microfluidic system that includes a substrate surface which displays two or more distinct microfluidic devices on the substrate surface. In certain embodiments, the microfluidic system includes a substrate surface with an array of microfluidic devices.

An “array” includes any two-dimensional or substantially two-dimensional (as well as a three-dimensional) arrangement of addressable regions, e.g., spatially addressable regions. An array is “addressable” when it has multiple devices positioned at particular predetermined locations (e.g., “addresses”) on the array. Array features (e.g., devices) may be separated by intervening spaces. Any given substrate may carry one, two, four or more arrays disposed on a front surface of the substrate. Depending upon the use, any or all of the arrays may be the same or different from one another and each may contain multiple distinct microfluidic devices. An array may contain one or more, including two or more, four or more, 8 or more, 10 or more, 50 or more, or 100 or more microfluidic devices. In certain embodiments, the microfluidic devices can be arranged into an array with an area of less than 10 cm², or less than 5 cm², e.g., less than 1 cm², including less than 50 mm², less than 20 mm², such as less than 10 mm², or even smaller. For example, microfluidic devices may have dimensions in the range of 10 mm×10 mm to 200 mm×200 mm, including dimensions of 100 mm×100 mm or less, such as 50 mm×50 mm or less, for instance 25 mm×25 mm or less, or 10 mm×10 mm or less, or 5 mm×5 mm or less, for instance, 1 mm×1 mm or less.

Arrays of microfluidic devices may be arranged for the multiplex analysis of samples. For example, multiple microfluidic devices may be arranged in series, such that a sample may be analyzed for the presence of several different analytes in a series of microfluidic devices. In certain embodiments, multiple microfluidic devices may be arranged in parallel, such that two or more samples may be analyzed at substantially the same time.

Aspects of the systems include that the microfluidic devices may be configured to consume a minimum amount of sample while still producing detectable results. For example, the system may be configured to use a sample volume of 100 μL or less, such as 75 μL or less, including 50 μL or less, or 25 μL or less, or 10 μL or less, for example, 5 μL or less, 2 μL or less, or 1 μL or less while still producing detectable results. In certain embodiments, the system is configured to have a detection sensitivity of 1 nM or less, such as 500 pM or less, including 100 pM or less, for instance, 1 pM or less, or 500 fM or less, or 250 fM or less, such as 100 fM or less, including 50 fM or less, or 25 fM or less, or 10 fM or less. In some instances, the system is configured to be able to detect analytes at a concentration of 1 μg/mL or less, such as 500 ng/mL or less, including 100 ng/mL or less, for example, 10 mg/mL or less, or 5 ng/mL or less, such as 1 ng/mL or less, or 0.1 ng/mL or less, or 0.01 ng/mL or less, including 1 pg/mL or less. In certain embodiments, the system has a dynamic range from 10⁻¹⁸ M to 10 M, such as from 10⁻¹⁵ M to 10⁻³ M, including from 10⁻¹² M to 10⁻⁶ M.

In certain embodiments, the microfluidic devices are operated at a temperature ranging from 1° C. to 100° C., such as from 5° C. to 75° C., including from 10° C. to 50° C., or from 20° C. to 40° C. In some instances, the microfluidic devices are operated at a temperature ranging from 35° C. to 40° C.

Systems of the invention may further include a signal processing module configured to receive signals from the reader output a result of a given assay, e.g., in the form of whether an analyte is present, the kinetics of an interaction of interest, etc. The signal processing module may be integrated into the system as a single device, or distributed from the other parts of the system where the signal processing module and other parts of the system are in communication with each other, e.g., via a wired or wireless communication protocol.

### Microfluidic Devices

Aspects of the invention further include microfluidic devices which include at least a PLE microchannel, e.g., as described above. Embodiments of the microfluidic devices may be made of any suitable material that is compatible with the microfluidic devices and compatible with the samples, buffers, reagents, etc. used in the microfluidic devices. In some cases, the microfluidic devices or components thereof, e.g., channels, are made of a material that is inert (e.g., does not degrade or react) with respect to the samples, buffers, reagents, etc. used in the subject microfluidic devices and methods. For instance, the microfluidic channels may be made of materials, such as, but not limited to, glass, quartz, polymers, elastomers, paper, combinations thereof, and the like.

The microfluidic devices of embodiments of the invention may include a number of distinct components. In some instances, the microfluidic devices include one or more sample input ports. The sample input port may be configured to allow a sample to be introduced into the microfluidic device. The sample input port may be in fluid communication with the PLE microchannel. In some instances, the sample input port is in fluid communication with the upstream end of the PLE microchannel. The sample input port may further include a structure configured to prevent fluid from exiting the sample input port. For example, the sample input port may include a cap, valve, seal, etc. that may be, for instance, punctured or opened to allow the introduction of a sample into the microfluidic device, and then re-sealed or closed to substantially prevent fluid, including the sample and/or buffer, from exiting the sample input port.

In some instances, the microfluidic devices include a concentration medium positioned upstream from the PLE microchannel. By “upstream” is meant positioned proximal to a source of a fluid flow. The concentration medium may be configured to concentrate the sample prior to the sample contacting the separation medium. The concentration medium may include a polymeric gel, such as a polymeric gel with a small pore size. For example, the concentration medium may include a polyacrylamide gel that has a total acrylamide content of ranging from 5% to 10%, such as from 5% to 9%, including from 5% to 8%, or from 5% to 7%. In some instances, the concentration medium has a total polyacrylamide content of 6%. In certain embodiments, the concentration medium includes a membrane, such as a size exclusion membrane. The small pore size of the concentration medium may slow the electrophoretic movement of the sample through the concentration medium, thus concentrating the sample before it contacts the separation medium. In some instances, the concentration membrane is configured to increase the concentration of the sample by 2 times or more, 4 times or more, 10 times or more, 25 times or more, 50 times or more, 100 times or more, 500 times or more, 1000 times or more, 2500 times or more, etc.

In some instances, the microfluidic device is configured to subject a sample to two or more directionally distinct flow fields. By “flow field” is meant a region where moieties traverse the region in substantially the same direction. For example, a flow field may include a region where mobile moieties move through a medium in substantially the same direction. A flow field may include a medium, such as a separation medium, a binding medium, a loading medium, etc., where moieties, such as buffers, analytes, reagents, etc., move through the medium in substantially the same direction. A flow field may be induced by an applied electric field, a pressure differential, electroosmosis, and the like. In some embodiments, the two or more flow fields may be directionally distinct. For example, a first flow field may be aligned with the directional axis of the separation flow path of the separation medium. The first flow field may be configured to direct the sample or analytes through the separation medium along the separation flow path. A second flow field may be aligned with the directional axis of the labeling flow path of the binding medium. In some instances, the second flow field is configured to direct the sample or analytes through the binding medium along the labeling flow path. The second flow field may be configured to direct the sample or analytes through the binding medium such that the analyte of interest contacts its specific binding member. In some instances, the second flow field is configured to direct a binding member through the binding medium along the labeling flow path. The second flow field may be configured to direct the binding member through the microchannel such that the binding member contacts its specific analyte of interest.

In certain embodiments, the microfluidic device is configured to subject a sample to two or more directionally distinct electric fields. The electric fields may facilitate the movement of the sample through the microfluidic device (e.g., electrokinetic transfer of the sample from one region of the microfluidic device to another region of the microfluidic device). The electric fields may also facilitate the separation of the analytes in the sample by electrophoresis (e.g., polyacrylamide gel electrophoresis (PAGE)), as described above. In some embodiments, the two or more electric fields may be directionally distinct. For example, a first electric field may be aligned with the directional axis of the separation flow path of the separation medium. The first electric field may be configured to direct the sample or analytes through the separation medium along the separation flow path. A second electric field may be aligned with the directional axis of the labeling flow path of the binding medium. In some instances. the second electric field is configured to direct the sample or analytes through the binding medium along the labeling flow path. The second electric field may be configured to direct the sample or analytes through the binding medium such that the analyte of interest contacts it specific binding member. In some instances, the second electric field is configured to direct a binding member through the binding medium along the labeling flow path. The second electric field may be configured to direct the binding member through the binding medium such that the binding member contacts its specific analyte of interest. As described above, in certain instances, the directional axis of the labeling flow path is orthogonal to the directional axis of the separation flow path. In these instances, the second electric field may be orthogonal to the first electric field.

In certain embodiments, the microfluidic device includes one or more electric field generators configured to generate an electric field. The electric field generator may be configured to apply an electric field to various regions of the microfluidic device, such as one or more of the separation medium, the binding medium, the loading medium, and the like. The electric field generators may be configured to electrokinetically transport the analytes and moieties in a sample through the various media in the microfluidic device. In certain instances, the electric field generators may be proximal to the microfluidic device, such as arranged on the microfluidic device. In some cases, the electric field generators are positioned a distance from the microfluidic device. For example, the electric field generators may be incorporated into a system for detecting an analyte, as described in more detail below.

In some cases, the downstream end of the transfer flow path is in fluid communication with a waste reservoir, such that the transfer flow path is configured to direct the moieties not of interest to the waste reservoir. In some cases, the downstream end of the transfer flow path is in fluid communication with a secondary analysis device, such that the transfer flow path is configured to direct the moieties that pass through the binding medium without binding to the binding member to the secondary analysis device for further characterization of the moieties. The secondary analysis device may include, but is not limited to, a UV spectrometer, and IR spectrometer, a mass spectrometer, an HPLC, an affinity assay device, and the like. In some instances, the secondary analysis device is included on the same substrate as the microfluidic device. In these embodiments, the microfluidic device and the secondary analysis device may be provided on a single substrate for the analysis of a sample by one or more different analysis techniques. In certain embodiments, the secondary analysis device is included as part of a system, where the system includes a microfluidic device and one or more separate secondary analysis devices. As described above, the microfluidic device and the secondary analysis device may be in fluid communication with each other, such that moieties that pass through the microfluidic device may be directed to the secondary analysis device for further characterization of the moieties.

In certain embodiments, the microfluidic device has a width ranging from 10 cm to 1 mm, such as from 5 cm to 5 mm, including from 1 cm to 5 mm. In some instances, the microfluidic has a length ranging from 100 cm to 1 mm, such as from 50 cm to 1 mm, including from 10 cm to 5 mm, or from 1 cm to 5 mm. In certain aspects, the microfluidic device has an area of 1000 cm² or less, such as 100 cm² or less, including 50 cm² or less, for example, 10 cm² or less, or 5 cm² or less, or 3 cm² or less, or 1 cm² or less, or 0.5 cm² or less, or 0.25 cm² or less, or 0.1 cm² or less.

In certain embodiments, the microfluidic device is substantially transparent. By “transparent” is meant that a substance allows visible light to pass through the substance. In some embodiments, a transparent microfluidic device facilitates detection of analytes bound to the binding medium, for example analytes that include a detectable label, such as a fluorescent label. In some cases, the microfluidic device is substantially opaque. By “opaque” is meant that a substance does not allow visible light to pass through the substance. In certain instances, an opaque microfluidic device may facilitate the analysis of analytes that are sensitive to light, such as analytes that react or degrade in the presence of light.

In some instances, microfluidic device includes a pore-limit electrophoresis (PLE) microchannel; and at least one of: a molecular weight ladder; and an assay reagent. In certain of these embodiments, the device includes a molecular weight ladder and the molecular weight ladder is positioned in a sample receiving region in fluid communication with the PLE microchannel. In certain instances, the device includes an assay reagent and is configured to introduce the assay reagent into the PLE microchannel following separation of sample components in the PLE microchannel, e.g., using multi directional flow paths and/or fields, such as described in PCT/US2010/035314; the disclosure of which is herein incorporated by reference. In some instances, the assay reagent is an enzyme substrate. In some instances, the assay reagent is a labeled reporter molecule. In some instances, the PLE microchannel includes a sample; a molecular weight ladder; and an assay reagent.

### Utility

The subject devices, systems and methods find use in a variety of different applications where evaluation of the interaction of first and second assay members is desired. In certain embodiments, the methods are directed to the detection of analytes, e.g., proteins (such as enzymes), nucleic acids or other biomolecules in a sample. Specific applications of interest include, but are not limited to: the detection of a set of biomarkers, e.g., two or more distinct protein biomarkers, in a sample, e.g., as described in PCT/US2010/035314, the disclosure of which is herein incorporated by reference; zymography methods, e.g., as described in greater detail below; and the like.

### Kits

Aspects of the present disclosure additionally include kits that have a microfluidic device as described in detail herein. The kits may also include at least one of: a molecular weight ladder; and an assay reagent. In some embodiments, the kits include additional components, e.g., a buffer, such as an electrophoretic buffer, a sample buffer, and the like, release agents, denaturing agents, refolding agents, detergents, detectable labels and labeled reporter agents (e.g., fluorescent labels, colorimetric labels, chemiluminescent labels, multicolor reagents, enzyme-linked reagents, avidin-streptavidin associated detection reagents, radiolabels, gold particles, magnetic labels, etc.), and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, Blu-Ray, computer-readable memory, etc., on which the information has been recorded or stored. Yet another means that may be present is a website address which may be used via the Internet to access the information at a removed site. Any convenient means may be present in the kits.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Experimental

I. Microfluidic Gradient-Gel Zymography Enables Quantitative Enzyme Activity Determination with Zeptomole Sensitivity

A. Introduction

Macroscale “zymography” techniques are widely applied to probe the activity of enzymes in situ following PA gel-based separation of protein samples (in both native and SDS-PAGE variants). In zymography, electrophoretic separation of protein constituents is directly followed by functional assays for enzymatic activity, often via diffusional exposure of protein bands to a low molecular weight substrate probe. An alternative approach utilizes prior immobilization of macromolecular substrates in the gel medium used for the electrophoretic separation. Zymographic methods enjoy wider use than western blotting for enzyme quantitation owing to the relatively low diversity of proprietary enzyme-specific antibodies currently available³¹.

The present work builds upon the sizing and pseudo-immobilization properties of pore-limit electrophoresis (PLE) by integrating PLE output with an enzyme activity assay to yield a microfluidic zymography format. PLE was conceived in the mid-1960s as a slab gel electrophoresis method allowing highly spatially resolved separation of protein bands from complex samples over a broad molecular weight range. Proteins electrophorese through a sieving gel whose pore-size decreases to dimensions on the order of the effective diameter of each macromolecule, leading to a progressive retardation of the analytes to a near stop. Conveniently, PLE yields a log-linear relationship between molecular weight and migration distance.

Described below is a two-stage pore limit electrophoresis with enzyme assay (PLENZ) that yields information about the size, amount, and activity of CIP enzyme from 500 pM stock solutions. This focused analysis is complemented by a proof-of-principle assay for horseradish peroxidase VI-A (HRP) in the Supporting Information. Localized on-chip fluorescent product generation provides significant signal amplification, making the assay sensitive to zeptomole amounts of enzyme using conventional CCD imaging. Our heterogeneous enzyme assay obviates the need for tailored protein immobilization chemistries by taking advantage of the physical pseudo-immobilization effect of PLE. In comparison to slab gel zymography, the microfluidic setting offers the benefits of reduced sample requirement and closer control over substrate access to a putative enzyme band as well as lower run times and higher detection sensitivity. The new PLENZ format forges a clear path towards quantitative single-input, multiple-output enzyme assays relevant to life science and clinical research.

B. PLENZ CIP Assay

1. Materials and Methods

a. Reagents. The UV photoinitiator 2,2′-azobis[2-methyl-N-(2-hydroxyethyl) propionamide] (VA-086) was purchased from Wako Chemicals (Richmond, Va.). 3-(trimethoxysilyl)propyl methacrylate (98%) for channel functionalization, 2-hydroxyethyl cellulose (HEC) for blocking channel flow, perchloric acid (70%, ACS grade) and hydrogen peroxide (30%, ACS grade) for dissolution of polyacrylamide gel from used devices, dimethyl sulfoxide (DMSO, molecular biology grade), methanol (ACS grade), glacial acetic acid (ACS grade), zinc chloride (reagent grade, >98%), N,N′-methylenebisacrylamide (bisacrylamide, >99%), horseradish peroxidase type VI-A (HRP, EC 1.11.1.7, ˜1,000 U mg⁻¹) and calf intestinal alkaline phosphatase (CIP, EC 3.1.3.1, ˜10,000 U ml⁻¹) were all from Sigma Aldrich (St. Louis, Mo.). Electrophoresis-grade glycine was purchased from Bio-Rad Laboratories (Hercules, Calif.). Magnesium chloride hexahydrate was from EMD (Darmstadt, Germany). Sodium cholate (99%), dithiothreitol (DTT, electrophoresis grade) and acrylamide (99%, electrophoresis grade) were all from Fisher Scientific (Hampton, N.H.). DiFMUP phosphatase substrate (6,8-difluoro-4-methylumbelliferyl phosphate, formal charge of −2 at pH 9.3) and its fluorescent product, DiFMU (6,8-difluoro-7-hydroxy-4-methylcoumarin, charge of −1) were from Invitrogen (Carlsbad, Calif.), as was the Amplex Red peroxidase substrate (approximately neutral at pH 7.4).

Unlabeled mouse monoclonal antibody to human C-reactive protein (aCRP, 150 kDa) and lactoferrin (LF, human, 80 kDa) were from Abcam (Boston, Mass.). Unlabeled carbonic anhydrase (CA, bovine, 30 kDa) was purchased from Sigma Aldrich. AlexaFluor (AF) 488-conjugated ovalbumin (OVA, chicken egg, 45 kDa) and trypsin inhibitor (TI, soybean, 21 kDa) were from Invitrogen. CIP, aCRP, LF and CA were fluorescently labeled with AF488 using commercial protein labeling kits (Invitrogen), purified using P-6 Bio-Gel columns (Bio-Rad), quantified by the method of Warburg and Christian (Layne, E. In Methods in Enzymology; Academic Press: New York, 1957; Vol. 3, pp 447-454) and stored at 4° C. until use. 10 mM stock solutions of DiFMUP, DiFMU and Amplex Red were prepared by dissolving the as-supplied solids in DMSO prior to storage in the dark at −20° C. For the CIP assay, six-protein ladder solutions consisting of 500 pM CIP; 250 nM aCRP, OVA and TI; and 500 nM LF and CA were made in a “run buffer” of the following composition: 75 mM glycine titrated with NaOH to a measured pH of 9.3, 1% sodium cholate, 1 mM MgCl₂, ZnCl₂, and DTT. Sodium cholate is a non-denaturing, anionic detergent with a low aggregation number that solubilizes CIP enzyme, improving loading of the enzyme into PLENZ devices and reducing the tendency for nonspecific adsorption to the gel matrix without compromising full substrate penetration. Glycine buffer was chosen due to its alkaline buffering range (pKₐ=9.8) and minimal observed inhibitory effects on intestinal phosphatases. DTT was also included as a mild reducing agent to further inhibit CIP aggregation. Divalent metal ions were provided in the buffer to prevent loss of structural and catalytic integrity of the enzyme.

b. PLENZ Device Fabrication

i. Glass chip preparation. Glass chips were fabricated using standard wet-etching techniques by Caliper Life Sciences (Hopkinton, Mass.). Four straight-channel devices per chip were isotropically etched to yield channels with a depth of 10 μm and width of 70 μm. Via wells provided fluidic access and were 2 mm in diameter. The PLENZ channels had a working length of 10.4 mm (well-edge to well-edge). Channels were cleaned and then functionalized using acrylate-terminated self-assembled monolayers as previously described⁴⁶ (details are provided in Supporting Information). Acrylamide (gel precursor) solutions consisted of 35% T (total acrylamide) with 12% C (percentage of acrylamide as crosslinker, bisacrylamide) and 10% T with 2.6% C in “gel buffer”. The gel buffer consisted of 75 mM glycine, 1 mM MgCl₂ and ZnCl₂. VA-086 was included in each solution to a concentration of 2 mg ml⁻¹ from a 0.1 mg μl⁻¹ suspension in deionized water. Care should be taken in handling acrylamide monomer, which is neurotoxic and readily absorbed through the skin.

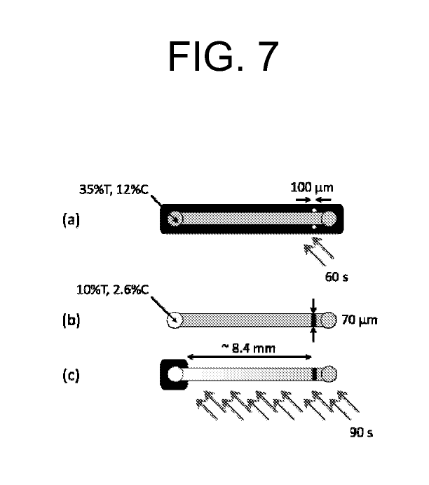

ii. Gradient gel fabrication. A multi-step photopolymerization process adapted from the literature was used to fabricate the PA pore-size gradient gels (Sommer, G. J.; Singh, A. K.; Hatch, A. V. Lab Chip. 2009, 9, 2729-2737; He, M.; Herr, A. E. Anal. Chem. 2009, 81, 8177-8184; Lo, C. T.; Throckmorton, D. J.; Singh, A. K.; Herr, A. E. Lab Chip. 2008, 8, 1273-1279), (FIG. 7). Gradients were generated using the “evolving boundary condition” approach of Lo et al., which does not rely on diffusion of monomer through the membrane structure to establish a linear gradient in gel composition. Following thorough degassing of the gel precursor solutions under vacuum with agitation, the 35% T solution was introduced to each channel and hydrodynamic flow temporarily halted by gently adding a layer of high viscosity 5% HEC solution to each via well. A ˜150 μm-long gel membrane was polymerized approximately three channel widths from one of the two via wells, as defined using a 100 μm by 500 μm slit in a plastic transparency mask (Fineline Imaging, Colorado Springs, Colo.). UV illumination in the 330-375 nm range was provided by the mercury lamp of an IX50 inverted fluorescence microscope (Olympus, Melville, N.Y.) directed through an ND25 filter, UV-permissive filter cube and 4× objective (UPlanSApo, N.A. 0.16). Membrane polymerization was achieved with an exposure time of 60s at a local UV intensity of approximately 20 mW cm⁻², as measured by a light meter at the plane of the chip (Mannix UV340 Light Meter, General Tools, New York, N.Y.). A face shield and personal protective equipment were used to prevent cutaneous exposure to UV light.

After photopolymerization of the small-pore membrane to establish one end of the PA gel gradient, the HEC solution was gently removed from each well with gel buffer. The 35% T gel precursor solution was then added to a 100 μl pipette tip press-fit into the via well proximal to the membrane. After degassing, the 10% T, 2.6% C acrylamide solution was similarly introduced at the opposite well. Chips were incubated in a dark, humid environment for 20 hr to establish a diffusion-generated 10-35% T gradient in gel composition along the channel. The 10% T loading well and first two channel widths were masked before final polymerization, providing for an unpolymerized loading region with no PA gel in the loading well. Each PA pore-size gradient was then polymerized by flood exposing the entire chip with UV light for 90s using an air-cooled 100 W mercury lamp (300-380 nm, UV intensity of ˜10 mW cm⁻², B-100AP Lamp, UVP, Upland, Calif.). Completed PLENZ chips were stored at room temperature in gel buffer until use (within 36 hours).

Removal of the gel matrix after use was achieved by overnight incubation of the entire chip in a 2:1 solution of 70% perchloric acid and 30% hydrogen peroxide heated to 75° C., allowing efficient recycling of glass chips (He, M.; Herr, A. E. Anal. Chem. 2009, 81, 8177-8184). As with solutions of strong oxidizing agents, this mixture should be handled with extreme care using appropriate personal protective equipment and other safety controls. Plastic-coated (exterior) glass bottles with vented caps were used for storage of waste solution, as one hazard of the solution includes gas generation leading to over-pressurization of sealed containers.

c. Apparatus and Imaging.

Chip imaging during separation and assay phases was conducted using a Nikon TE2000-E inverted fluorescence microscope equipped with motorized stage (Applied Scientific Instrumentation, Eugene, Oreg.), filter wheel and shutter systems (Uniblitz, Rochester, N.Y.). All channel images were taken at exposure times of 70 ms or 200 ms (as indicated) using a 10× objective (PlanAPO, N.A. 0.45) and a 2048×2048 interline CCD camera (CoolSNAP K4, Princeton Instruments, Trenton, N.J.). 4×4 pixel binning was used to minimize signal noise. Labeled proteins were imaged via a FITC HQ filter cube (Ex. 460-500 nm, Em. 510-560 nm); hereafter termed the “green fluorescence” channel. DiFMUP and DiFMU were imaged using a UV-2E/C filter cube (Ex. 325-375 nm, Em. 435-485 nm); hereafter termed the “blue fluorescence” channel. Fluorescence images of the axial protein, DiFMUP and DiFMU distributions were collected at 1 min intervals as required during the PLE separation and assay phases. Sets of 14 overlapping exposures were digitally stitched together from a 600 μm by 13.3 μm region of interest (ROI) within the channel footprint to yield spatial electropherograms of green and blue fluorescence covering 8.4 mm of the channel length. Background subtraction was carried out using an identical ROI lying outside of the separation channel.

Image analysis was conducted using ImageJ software (National Institutes of Health, Bethesda, Md., http://rsbweb.nih.gov/ij/). Nonlinear least squares fitting was carried out via the Marquardt-Levenberg algorithm using gnuplot software (http://www.gnuplot.info/). Surfaces and electropherograms generated from image intensity data were visualized using SpOd plotting and graphics software (courtesy of Dr. Mark R. Titchener, University of Auckland, New Zealand). All species were electrophoretically driven through the gradient gel devices using a programmable high voltage power supply via platinum wire electrodes directly inserted into 5-10 μl pipette-tip sample reservoirs (1275 LabChip Controller, Caliper Life Sciences, Hopkinton, Mass.).

To convert arbitrary fluorescence data to corresponding standard concentration units, calibration curves were generated from CCD images of open microchannels filled with free solution dilution series of the protein ladder (0.001-0.3x), DiFMUP (50-2000 μM), and DiFMU (0.1-70 μM) given the known volume of the ROI locus along the channel axis. These curves were found to be linear over the indicated concentration ranges (R²>0.99). Excluded volume effects on local species concentrations caused by the presence of gel matrix in the PLENZ channel were assumed to be negligible. Gold-standard assays of enzyme activity were conducted at a CIP concentration of 30 pM in run buffer using a Safire2 fluorescence microplate reader (Tecan Group, Mannedorf, Switzerland).

C. PLENZ HRP Assay

1. Chip functionalization. To clean and condition glass channel surfaces prior to gel polymerization, all channels were incubated with 1M NaOH for 5 min followed by two washes with deionized water. Vacuum was used to purge the channels with air between introductions of each solution by capillary action. To ensure firm attachment of the PA gel in the channel, all channel surfaces were functionalized using acrylate terminated self-assembled monolayers by incubating channels with a 2:3:5 mixture of 3-(trimethoxysilyl)propyl methacrylate: glacial acetic acid: deionized water for 30 min. Washing with methanol and DI water was repeated twice, with final drying achieved by vacuum purging.

2. HRP assay. Details of chip fabrication and experimental procedure for the horseradish peroxidase assay were identical to those given for the CIP assay in all but the following areas. The 1x gel buffer composition was 25 mM Tris-HCl titrated to pH 7.4 with NaOH; this solution supplemented with sodium cholate at 1% by weight served as the run buffer. The HRP substrate solution consisted of 100 μM Amplex Red (approx. neutral charge at pH 7.4, having an orange fluorescent resorufin product) and 6 μM H₂O₂ (also approx. neutral at pH 7.4) in run buffer (Towne, V.; Will, M.; Oswald, B.; Zhao, Q. Anal. Biochem. 2004, 334, 290-296). N.B. DTT was not included in the run buffer as resorufin is unstable in its presence. The protein sample was identical in composition with respect to the molecular weight markers, but included HRP type VI-A (unlabeled) at 100 pM instead of CIP.

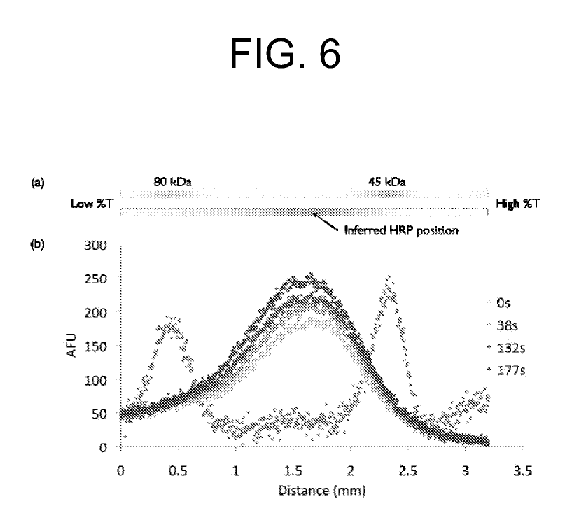

FIG. 6 shows a 3.2 mm portion of a PLENZ channel surrounding the site of resorufin production by HRP, with 80 and 45 kDa marker peaks revealed in the green fluorescent channel. Measurements were taken using an Olympus IX70 fluorescence microscope with 4× UPlanFl objective (N.A. 0.13), 1392×1040 interline CCD camera (CoolSNAP HQ2, Princeton Instruments), XF100-3 (Ex. 445-495 nm, Em. 510-580 nm, green fluorescence) and XF111-2 (Ex. 525-555 nm, Em.>575 nm, orange-red fluorescence) filter cubes (Omega Optical, Brattleboro, Vt.), manual x-y translation stage and excitation shutter. The protein sample was loaded at an applied current of 0.2 μA (measured voltage of 240V) for 60s and subsequently driven through the PLENZ gradient with run buffer for 20 min. The substrate mixture was then introduced via the 10% T loading well for a further 20 min under the same electrophoresis conditions, at which point the flow was halted. Resorufin production by HRP was imaged using sequential 200 ms exposures under orange-red fluorescence after recording the initial distribution of marker peaks via the green fluorescence.

The position of resorufin generation places the HRP VI-A enzyme at a molecular weight of approximately 50-55 kDa. From vendor information, the nominal weight of HRP is around 44 kDa, of which 9.4 kDa is carbohydrate. The deviation in position of the observed activity peak from the expected position is corroborated by literature reports of macroscale SDS-PAGE separation and zymography of HRP VI (Loustau, M. N.; Romero, L. V.; Levin, G. J.; Magri, M. L.; Lopez, M. G.; Taboga, O.; Cascone, O.; Miranda, M. V. Process Biochem. 2008, 43, 103-107; Kuhtreiber, W. M.; Serras, F.; van den Biggelaar, J. A. M. Development. 1987, 100, 713-722), and is likely attributable to the high degree of glycosylation of the enzyme.

3. Use of current control strategy. Interestingly, the applied voltage across PLENZ devices was observed to have little influence on the migration behavior of analytes, despite the convention of plotting PLE peak migration distances against volt-hours on the abscissa (Sommer, G. J.; Singh, A. K.; Hatch, A. V. Lab Chip. 2009, 9, 2729-2737; Slater, G. G. Anal. Chem. 1969, 41, 1039-1041). Indeed, the electric field ranged between 50 and 300 V cm⁻¹ for a common applied current of 0.3 μA, even though peak velocity profiles as a function of channel distance were found to be repeatable (data not shown). A lack of strict dependence of peak migration on the product of voltage and time also appears in data presented by Sommer et al. over the range of 0-50 Vh (i.e. in the pre-pore limit regime) (Sommer et al. id). We hypothesize that this departure from theory arises from non-uniformity in the electric field, and in particular, to the discontinuity that the photopatterned membrane presents to the transport of charged species. Fuxman et al. suggest that the pore-size distribution in photopatterned PA structures can be affected by gradients in monomer concentration that emerge during polymerization, leading to especially small pore-size at their edges (Fuxman, A. M.; McAuley, K. B.; Schreiner, L. J. Chem. Eng. Sci. 2005, 60, 1277-1293). This phenomenon has recently been observed and characterized for PA gel membranes in microfluidic channels (Hou, C.: Bioengineering, University of California, Berkeley, Calif. Personal communication, 2009). Concentration polarization can also be observed when current is applied across high-percentage PA membranes, leading to a progressive increase in membrane electrical resistance over time (Hatch, A. V.; Herr, A. E.; Throckmorton, D. J.; Brennan, J. S.; Singh, A. K. Anal. Chem. 2006, 78, 4976-4984). The 35% T membranes used in the PLENZ device to halt hydrodynamic flow during gradient formation therefore constitute regions of high and variable resistance to the passage of charged species. As a result, the membrane-associated resistance can dominate the observed well-to-well voltage drop, making it difficult to measure the true electric field in the separation gel. Use of a constant current rather than voltage ensures a consistent electric field in the separation region given repeatable pore characteristics between devices, while allowing for variability in the voltage drop associated with the membrane structure.

D. Results and Discussion

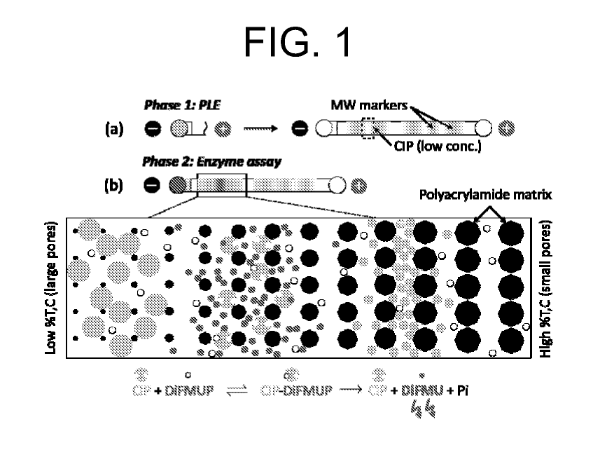

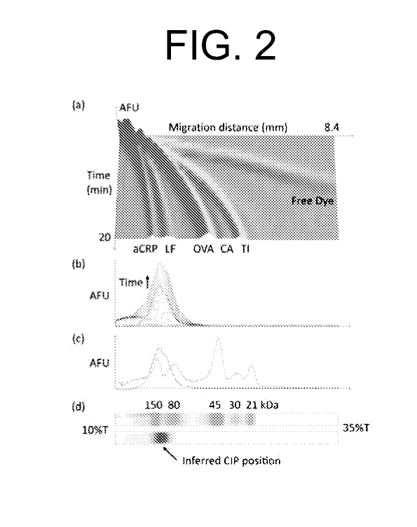

PLENZ consists of two integrated phases that together report enzyme molecular weight, amount, and activity—phase 1, the PLE protein sizing step (FIG. 1a) and phase 2, the enzyme assay step (FIG. 1b). The two phases are not independent, as fluorescent ladder (marker) species sized in phase 1 (FIG. 2a) allow determination of the molecular weight of an unlabeled and/or low-abundance enzyme through the axial location of fluorescent product generation in phase 2 (FIGS. 2b, c, d). In the case of CIP, the minimal overlap in emission spectra of green AF488 and blue DiFMU enabled concurrent imaging of the marker proteins and product generation (FIG. 2d). This section details development, optimization, and the resulting performance of PLENZ.

1. PLENZ Phase 1: Protein Sizing via PLE

As described previously for PLE (Sommer, G. J.; Singh, A. K.; Hatch, A. V. Lab Chip. 2009, 9, 2729-2737), the migration distance x(t) of a given analyte is logarithmically related to the product of the applied voltage and run time according to Equation 1.

x(t)˜In(1+Vt) (1)

Thus, a rapid initial migration period as the analytes travel through the gradient gel is followed by progressive slowing of the constituents towards a pseudo-immobilized state (at which point the “pore limit” is said to have been reached). To arrive at the description of PLE given in Equation 1, the applied electric field is assumed to be spatially uniform and the gel composition (% T and C) is assumed to increase linearly along the separation axis.

In the PLE sizing phase, a protein sample was electrophoretically introduced at the 10% T, 2.6% C end of PLENZ devices by applying a constant current of 0.3 μA for loading times of 30-105 s. Five protein species were used as molecular weight markers with a sixth species (CIP) acting as the enzyme target. After the ladder was loaded into a short open-channel loading region, the solution in the well was replaced with run buffer. The 20 min PLE separation phase was then initiated by applying constant current, again at 0.3 μA, while imaging the green fluorescence along the separation axis. FIG. 2a captures the resulting time-evolution of the molecular weight ladder peak structure generated by PLE.

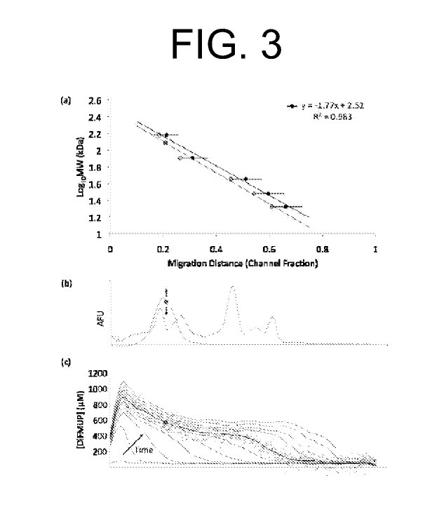

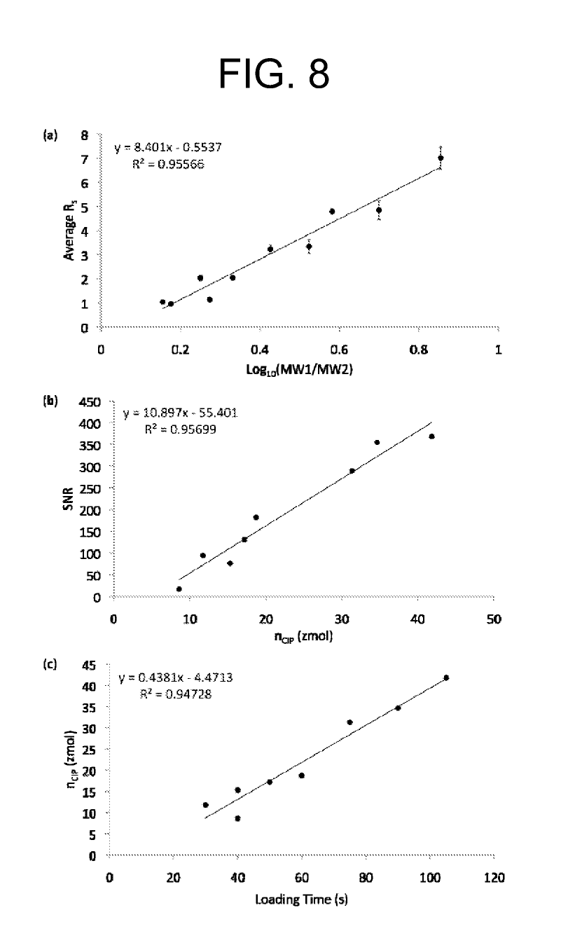

FIG. 3a demonstrates that species molecular weight was linearly related to migration distance on a log-linear scale in PLENZ devices (R²=0.98), as anticipated. The standard deviations in migration distance for the 5 marker protein constituents were between 4.9% and 6.2% of the total channel length (N=8 devices). Although the variations in species migration distance between devices were acceptable; the use of a sizing ladder in each gel fully corrects for these as is standard practice in PAGE slab-gels. Judging by extrapolation of the log weight versus migration distance plot to the boundaries of the device for the PLE conditions used here, the 10% T to 35% T gradient gel can accommodate a range in protein size from ˜6 kDa to ˜330 kDa. Analysis of the ladder electropherograms gave an average protein peak capacity of 16.0±0.3 peaks (error reported as SD, N=6) and a minimum resolvable pair-wise molecular weight ratio of ˜1.5 (Supporting Information, FIG. 8a). At 1.43, the smallest ratio for the protein ladder is comparable (CA, 30 kDa : TI, 21 kDa).

The PLE separations developed here were consistent with results presented previously, which showed an increase in the peak capacity for fluorescently-labeled transferrin from ˜17-30 peaks between 13 V-hr (33 min at 24V applied) and 105 V-hr (4.4 hours) as the protein approached its pore limit at ˜100 V-hr in a 5 mm micro-PLE channel (Sommer, G. J.; Singh, A. K.; Hatch, A. V. Lab Chip. 2009, 9, 2729-2737). These results support the theoretical expectation that the protein peak resolution increases (to a limit) with the product of time and applied electric field in the separation region (Sommer, G. J.; Singh, A. K.; Hatch, A. V. Lab Chip. 2009, 9, 2729-2737). Upon further comparison, it becomes clear that the separations conducted in this work have not been carried to full completion due to the prohibitive run times necessary. To ensure a reasonable assay time given that an upper bound on the applied voltage is enforced by the stability of the gel matrix (˜300 V cm⁻¹ at 0.3 μA), the transition to the activity assay phase is best initiated before the proteins arrive at their absolute pore limits, but after sufficient resolution of all species is established. For PLENZ devices, a separation time of 20 min was found to adequately address the tradeoff between the resolution of ladder constituents (and therefore the accuracy of enzyme sizing) and the assay duration.

As reviewed above, the applied voltage across PLENZ devices was observed to have little influence on the migration behavior of analytes, despite the convention of plotting PLE peak migration distances against volt-hours on the abscissa. Indeed, the electric field ranged between 50 and 300 V cm⁻¹ for a common applied current of 0.3 μA, even though peak velocity profiles as a function of channel distance were found to be repeatable (data not shown). We attribute the fluctuation in applied potential to the presence of the small pore-size membrane at the terminus of the PLE channel. Thus, a current-control strategy was instead employed to ensure a consistent electric field in the separation region (device-to-device) as detailed in the Supporting Information.

2. PLENZ Phase 2: In Situ Enzyme Activity Assay

a. Substrate transport and distribution. Post-electrophoresis enzyme assays in macroscale slab-gels are semi-quantitative at best (Hattori, S.; Fujisaki, H.; Kiriyama, T.; Yokoyama, T.; Irie, S. Anal. Biochem. 2002, 301, 27-34; Kaberdin, V. R.; McDowall, K. J. Genome Res. 2003, 13, 1961-1965), mainly due to the inability to quantify and control the substrate concentration at a given time and position in the gel. For example, when a small substrate molecule is incubated with the slab-gel, enzyme-mediated conversion of substrate to product is confounded by the kinetics of substrate diffusion from the gel surface to the true reaction site. In contrast, PLENZ uses electrophoresis to direct a small substrate along the separation axis to pseudo-immobilized proteins after the PLE phase. The low background fluorescence of DiFMUP substrate provides sufficient signal to study its transport through the PLE gel, thereby allowing quantitation of the substrate concentration within the gel as a function of time. The substrate(s) to be introduced need not necessarily be charged, as is demonstrated by the HRP assay reported above. This assay relies on electrophoretic transport of fluorogenic Amplex Red and H₂O₂ (neutral under the assay conditions) to the site of HRP pseudoimmobilization in the presence of the anionic surfactant sodium cholate. The underlying mechanism here is likely related to that operating in micellar electrokinetic chromatography (MEKC), in which the electrophoretic mobilities of neutral species are modified by their association with charged surfactant molecules (Otsuka, K.; Terabe, S. Mol. Biotechnol. 1998, 9, 253-271). Certainly, alternative modes of substrate introduction taking advantage of the microfluidic setting to allow quantitative kinetic analysis may be performed with routine modifications of the PLENZ chip geometry, e.g., low characteristic length substrate diffusion into the gel from a neighboring free-solution channel.

FIG. 3c tracks the progression of a DiFMUP substrate front as the interface electrophoreses through a PLENZ device over a period of 10 min (i=0.3 μA). Blue fluorescence along the channel length was imaged using a series of 200 ms exposures collected every minute, and the resulting axial fluorescence intensity profiles were smoothed using a Savitsky-Golay least squares fitting routine (Savitsky, A.; Golay, M. J. E. Anal. Chem. 1964, 36, 1627-1639). The decreasing concentration profile away from the low % T end of the device suggests that the electric field increases as the pore-size decreases, skewing the substrate distribution away from the disperse piston-like front expected under the convection-diffusion equation. Also, the DiFMUP concentration established at a given channel position after passage of the initial front is generally higher than the 300 μM nominal concentration in the loading well due to the drop in electrophoretic velocity of DiFMUP between the free solution and gel matrix under the constraints of conservation of mass. Thus, substrate loading into the gradient gel has complex dependencies on time and channel position that warrant direct measurement of DiFMUP concentration, as is applied for the substrate-limitation experiments in this work.

b. In situ measurement of enzyme activity and kinetic parameters. Subsequent to PLE, an assay for enzyme activity was performed on the set of pseudo-immobilized ladder proteins. The two-parameter Michaelis-Menten relationship is a parsimonious model of enzyme activity that captures the first-order generation of product at low substrate concentrations (i.e. limiting substrate availability), with a gradual shift towards zeroth-order conversion as the substrate concentration increases to saturation (i.e. limiting enzyme active site availability). Although more detailed models of CIP activity have been put forward, the Michaelis-Menten equation has offered reasonable correspondence to kinetic data arising from several microfluidic platforms utilizing CIP as a model analyte. Equation 2 relates the product generation rate [symbol] (the “activity”, in unconventional units of mol s⁻¹ rather than M s⁻¹) to the substrate concentration s (which is assumed to be locally constant across a given enzyme peak width). The Michaelis constant Kₘ is the substrate concentration at which [symbol] reaches half of its maximum value, [symbol] , where nₑ is the total amount of enzyme present in the system and k_{cat} is known as the turnover number. In this paper, [symbol] (s⁻¹) is [symbol] normalized by nₑ.

[symbol]

(2)

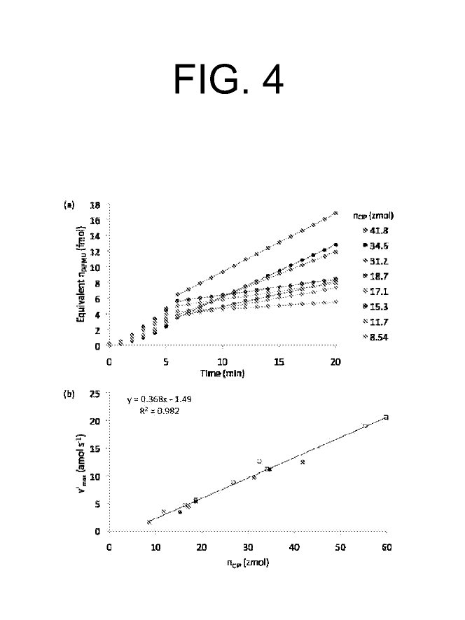

c. Enzyme-limiting conditions. The expected linear relationship between the rates of product DiFMU generation under approximately saturating DiFMUP concentrations (slopes of solid lines in FIG. 4a) and the amounts of enzyme loaded is confirmed in FIG. 4b, which shows an R² value of 0.98 across measurements conducted in 15 PLENZ devices on two separate days. In this work, the amount of enzyme loaded was inferred from the total area under the green fluorescence profile during the separation phase (determined by numerical integration) in conjunction with a protein ladder calibration curve. Inherent in this method is the assumption that the composition of the protein aliquot loaded into a PLENZ device was equivalent to that of the bulk solution in the loading well. To achieve a saturating substrate concentration at the site of reaction following ladder separation, a 1000 μM DiFMUP solution was electrophoretically introduced for 6 min at an applied current of 0.3 μA. The “stopped-flow” condition after 6 min was employed to fix the substrate concentration profile in the channel and remove the obfuscating influence of substrate/product transport from the loading well. The assumption of saturating conditions eliminated the need to measure the substrate profile prior to loading and separation of the ladder sample, and is validated by the fact that the local DiFMUP concentrations resulting from introduction of such a high nominal concentration solution were on the order of 3-5 times higher than the measured Kₘ of CIP in the PLENZ device. The rate of change in the total area under the blue fluorescence signal collected using sets of 70 ms exposures gave a lumped indication of DiFMU generation in each device, which occurs in a nonlinear fashion as a function of channel distance because the CIP enzyme adopts an approximately Gaussian distribution within the PLENZ gradient. Using the total blue area eliminated much of the complexity of this spatially non-uniform system, leading to the observed linear increases in the blue fluorescence areas after the point at which the electric field was halted in each device (again, solid lines in FIG. 4a).

d. Limits of CIP enzyme detection. The minimum amount of CIP enzyme detectable under DiFMUP saturation using the PLENZ system (the “limit of detection”, LOD), was evaluated by extrapolating a plot of the blue fluorescence signal:noise ratio (SNR) to a lower threshold value of 3. The SNR was defined as the maximum blue fluorescence at the reaction site less the “shoulder” fluorescence at the entrance to the loading well (the signal), divided by the standard deviation of the brightness over a channel region in which no reaction was observed (the noise). This procedure yields a mass LOD of 5.4 zmol (FIG. 8b). A consistent but less conservative value is offered by the x-intercept of FIG. 4b at ˜4.0 zmol (0.57 fg, or on the order of 2500 CIP molecules). The approximate PLENZ LOD of 5 zmol (0.7 fg) compares favorably with both the low picogram sensitivities reported for macroscale zymographic techniques (Hattori, S.; Fujisaki, H.; Kiriyama, T.; Yokoyama, T.; Irie, S. Anal. Biochem. 2002, 301, 27-34; Kaberdin, V. R.; McDowall, K. J. Genome Res. 2003, 13, 1961-1965) and the value of 52 zmol reported by Wu et al. for alkaline phosphatase in a capillary electrophoresis (CE) assay system (Wu, D.; Regnier, F. E. Anal. Chem. 1993, 65, 2029-2035). However, greater sensitivity is achievable, as has been demonstrated by the application of laser-induced fluorescence detection in a CE system to study CIP kinetics down to the single molecule level (Craig, D. B.; Arriaga, E. A.; Wong, J. C. Y.; Lu, H.; Dovichi, N. J. J. Am. Chem. Soc. 1996, 118, 5245-5253).

Practically, the amount of enzyme introduced into PLENZ devices was manipulated in an “open-loop” fashion using the loading time (FIG. 8c), reinforcing the ability of PLE to concentrate analytes from dilute samples. To this end, Sommer et al. report concentration factors up to 4-5 orders of magnitude (id). Nevertheless, the fidelity of the molecular weight information provided by spatial resolution of the marker protein peaks is adversely affected by extended loading times (as the axial length of the injected plug increases relative to the channel length available for separation). This tradeoff between sample preconcentration and molecular weight determination can be resolved by augmenting PLENZ with a second polyacrylamide exclusion membrane optimized for protein enrichment (Hatch, A. V.; Herr, A. E.; Throckmorton, D. J.; Brennan, J. S.; Singh, A. K. Anal. Chem. 2006, 78, 4976-4984).

e. Enzyme sizing during assay. In FIG. 2d, visual comparison of pseudo-color images of the PLENZ green and blue fluorescence channels illustrates that the position of enzyme activity can be compared to a fluorescent protein ladder and, thus, directly imparts molecular weight information for the low-abundance enzyme species. The positions of maximum enzyme activity for a set of eight PLENZ devices yield a molecular weight of 127±2.3 kDa (1.8%) for CIP by comparison to the corresponding marker protein migration relationships (% RSD in parentheses, see FIGS. 3a, b). This value compares well with the nominal weight of approximately 140 kDa reported in the literature (Durchschlag, H.; Christi, P.; Jaenicke, R. Progr. Colloid Polym. Sci. 1991, 86, 41-56), giving an accuracy of within 10%.

DiFMU is a small molecule and, hence, diffuses away from the reaction site over time. The diffusive broadening of DiFMU erodes the resolution of the position information that guides inference of enzyme molecular weight. Thus, we take the time derivative of the blue fluorescence at the start of the stopped-flow period of the assay phase using quadratic fits to the sets of brightness values at each ROI pixel along the channel length. This measure represents the enzyme activity as a function of distance in a more intuitive manner while minimizing the impact of apparent reaction rates caused by diffusional transport of DiFMU from neighboring pixels as local concentration gradients increase over time. The peak capacity of the blue fluorescence time derivative is approximately 6.5 for the case study in FIG. 2b, indicating a modest potential for multiplexed assays in the same gel even in the absence of a spectral encoding strategy providing for enzyme products with distinct emission properties. The fact that this capacity is smaller than the averaged value of 16 for the protein bands reflects an intuitive upper bound on the product peak resolution that is set by the underlying protein distribution. In other words, even in the absence of product diffusion, the resolution of multiple enzyme reactions in a microchannel can only ever be as good as the “parent” enzyme resolution.

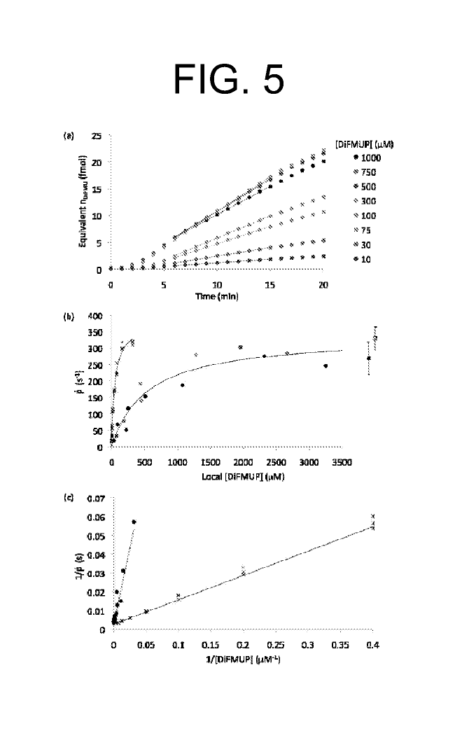

f. Substrate-limiting conditions. The saturating relationship between enzyme activity and local substrate concentration prescribed by the Michaelis-Menten model is illustrated for CIP in the PLENZ platform in FIG. 5. The reaction rates have been normalized by the amount of enzyme loaded to isolate the effect of substrate concentration, which was varied by introducing 10-1000 μM solutions of DiFMUP into separate PLENZ devices for 6 min at 0.3 μA at the beginning of the assay phase. Additionally, the substrate entry profile for each device was directly measured prior to the separation and assay phases. For this step, blue fluorescence imaging of the channel was conducted using an exposure time of 200 ms after a 6 min period of continuous introduction of a 300 μM solution of DiFMUP at 0.3 μA. The substrate was then removed from the channel using a 5 min wash with run buffer to prevent premature contact between enzyme and substrate during the PLE phase. This procedure allowed post hoc inference of the local substrate concentration in the region of maximum enzyme activity observed during the later assay phase, under the assumption that the DiFMUP entry profile could be linearly scaled to suit the appropriate nominal (via well) concentration of DiFMUP applied.

Again, DiFMU accumulation during the assay phase occurred linearly after the electric field was halted at the 6 min mark (solid lines in FIG. 5a). The Michaelis-Menten plot stemming from the corresponding rates and local substrate concentrations across two separately calibrated experiments is fit well by the saturating two-parameter model, giving a k_{cat} of 336 s⁻¹ and Kₘ of 545 μM (FIG. 5b). A similar experiment run in triplicate on a fluorescence microplate yielded a comparable k_{cat} of 376±10 s⁻¹, but a greatly reduced Kₘ of 49±1.4 μM. The two datasets from FIG. 4b confer precision information on the PLENZ data, giving k_{cat} values of 268±50 s⁻¹ and 329±35 s⁻¹ (corresponding RSD values are 19% and 11% respectively). The relatively smaller k_{cat} values associated with these data are expected, given that they arise from an assumption of saturating local DiFMUP concentration rather than from a non-linear fit that outputs an asymptotic value for k_{cat}. A summary of the kinetic data arising from the PLENZ and microplate measurements is presented in Table 1.

TABLE 1
Kinetic parameters for CIP enzyme from
microplate and PLENZ experiments^{a}.

| | k_{cat} (s⁻¹) | Kₘ (μM) |
|---|---|---|
| Microplate, N = 3 replicates | 376 ± 10 (2.7%) | 49 ± 1.4 (2.9%) |
| PLENZ - rate vs. [DiFMUP] | 336 | 545 |
| PLENZ - rate vs. n_{CIP} replicate 1, | 329 ± 35 (11%) | — |
| N = 7 devices | | |
| PLENZ - rate vs. n_{CIP} replicate 2, | 268 ± 50 (19%) | — |
| N = 8 devices | | |

^{a}Errors are reported as standard deviation with % RSD in parentheses. N.B. all experiments conducted in 75 mM glycine buffer, pH 9.3 with 1% sodium cholate, 1 mM MgCl₂, ZnCl₂, and DTT.

The similarity in the k_{cat} values measured via PLENZ and fluorescence microplate suggests that physical pseudo-immobilization of CIP in the PLENZ gel gradient does not destroy native enzyme activity, as is often the case with covalent enzyme immobilization to channel surfaces or packings. The Lineweaver-Burk plot in FIG. 5c reinforces this similarity in k_{cat} as a convergence of the two relationships upon approximately the same y-intercept. Here the transformed data are shown with the previously fitted Michaelis-Menten functions rather than with lines of best fit due to the lack of statistical rigor of the latter method. The greatly increased Kₘ in the PLENZ device without a drop in k_{cat} is reminiscent of competitive inhibition, but in this case can be attributed to the reduced molecular diffusivity of DiFMUP in the gel matrix as compared to the free-solution reaction state in the microplate well. Certainly, diffusional effects on the observed Kₘ are to be expected following physical entrapment of CIP in the PLENZ polyacrylamide matrix. Encouragingly, however, the results reported here show that direct loss of activity in a way that alters the observed k_{cat} is bypassed as the enzyme is electrophoretically introduced after gel polymerization.

E. Conclusions

We report a robust analytical method for determining enzyme molecular weight and activity information from a dilute aliquot of a heterogeneous protein sample. A PLE system of minimal complexity was optimized to allow rapid physical pseudo-immobilization and sizing of sample proteins in series with a fully quantitative in situ and label-free enzyme assay using microfluidic technology. The use of an electrophoretic physical immobilization strategy brings benefits over covalent immobilization in terms of the fidelity of the enzyme activity information collected and the fact that sample pre-functionalization is not necessary. Electrophoretic transfer of CIP into the microfluidic gel matrix was found to have little effect on its maximum activity, alleviating (but not eliminating) the bias that covalent immobilization strategies impose on the quantification of kinetic data. PLENZ also exhibits excellent sensitivity to enzyme activity with zero dead-volume due to the ability to load an exceedingly small protein aliquot directly onto the gradient gel without the need for accessory channels upstream of the injection point. The PLENZ-based horseradish peroxidase assay further demonstrates the ability of this technique to be adapted to various buffer systems, and to be applied to analytes having substrates with neutral charge.

This microfluidic zymographic platform will prove a useful addition to the molecular biology toolbox, particularly where it is of interest to perform functional screening of gene expression in a highly parallelized fashion. Such a platform will also be useful in functional proteomic studies, in which enzyme identification by mass spectrometry and amino acid sequencing is often performed downstream of an in situ PAGE separation and assay technique (Manchenko, G. P. Handbook of Detection of Enzymes on Electrophoretic Gels, 2nd ed.; CRC Press: New York, 2003; Patterson, S. D. Anal. Biochem. 1994, 221, 1-15). A multiplexed PLENZ system in which a panel of enzymes in an aliquot of biological fluid can be enriched, spatially resolved, and assayed in a single polyacrylamide gradient gel is currently under development, as are efforts to maximize the effective separation resolution through rational spectral encoding of fluorogenic substrates. Demonstrating the ability of PLENZ devices to process complex biological samples with multiple activity readouts from spatially and/or spectrally distinct enzyme bands will ultimately be directed towards improvements over gold-standard screening methods employed in drug discovery, clinical diagnostics and biocatalyst engineering. The analytical techniques developed here for PA gel-based fluorescence detection of biocatalytic activity will also complement progress towards a microscale immunoblotting paradigm, which has recently been reported by our group (He, M.; Herr, A. E. Anal. Chem. 2009, 81, 8177-8184).

II. Microfluidic Pore Limit Electrophoresis for Immunoassays and Binding Assays (PLE-IA)

A. Introduction

Reported here is a simple, low cost, adaptable platform that can perform protein sizing and quantitative analysis by immunoassay or other binding assays. PLE-IA involves the pseudo immobilization of antibody in a polyacrylamide gel with a linear gradient in pore size which is easier to perform and customize. Reported below is quantitative PLE-IA using three antibody-antigen pairs. Sizing and identification of target protein from a mixture is also proved below. The method reported below can be developed to fully automated, high throughput tools with broad applications.

B. Methods

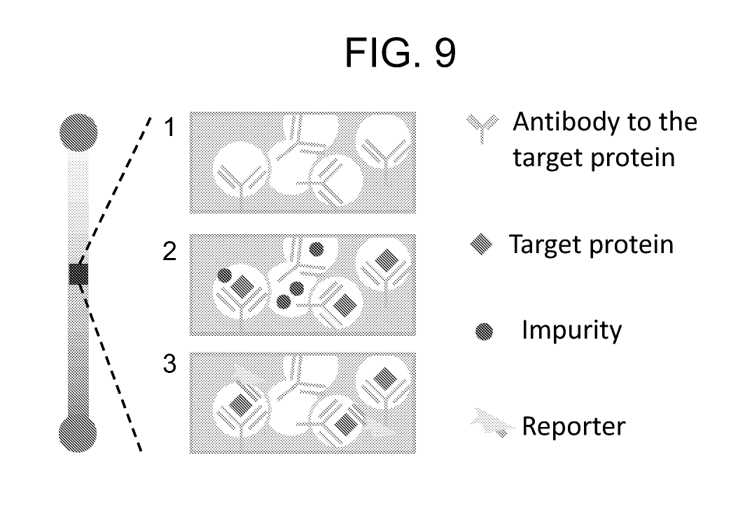

The microfluidic chips and the procedure for fabrication the gradient gel were the same as in PLENZ, e.g., as described in Example 1 above. FIG. 9 shows the principle of PLE-IA. First, antibody will be immobilized in the channel at a position where the pore size is small enough that the antibody is close to stop. Then the sample containing target protein will be injected into the channel, the target protein should has a smaller size than the antibody, so that it can pass through the antibody band. In the last step, a reporter will be used to detect any target protein that is captured by the antibody. The reporter can be the Fab fragment of a second antibody, aptamers, lectins or RNA for specific detection of the captured target protein or a fluorescent dye that labels all the protein in the channel.

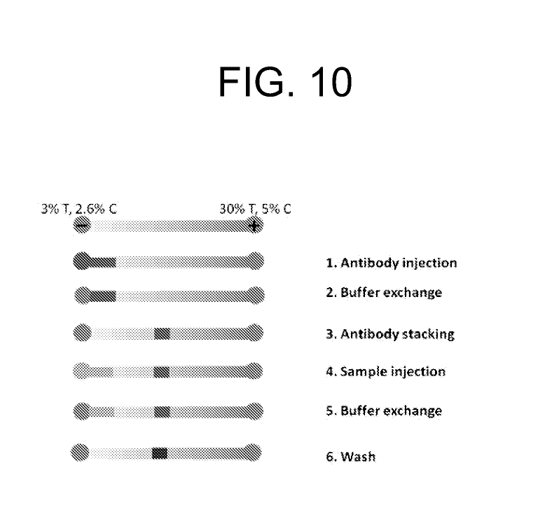

FIG. 10 shows the general steps involved in PLE-IA. A plug of the antibody to the target protein was first injected into the channel from the low percentage (big pore size) end. Then the antibody solution in the reservoir was replaced with buffer, followed by transportation of the antibody to the middle of the channel through electrophoresis migration. Next, sample solution containing the target protein was injected into the channel. The time of injection can be varied to control the amount of protein injected into the channel. Eventually, the unbound protein migrated away from the antibody band and stopped at a position determined by its size. The size information of the protein can be read out by comparing the position of the unbound protein to protein ladder ran parallel in a distinct channel.

C. Results

1. Linear Relationship Between Log Molecular Weight and Migration Distance

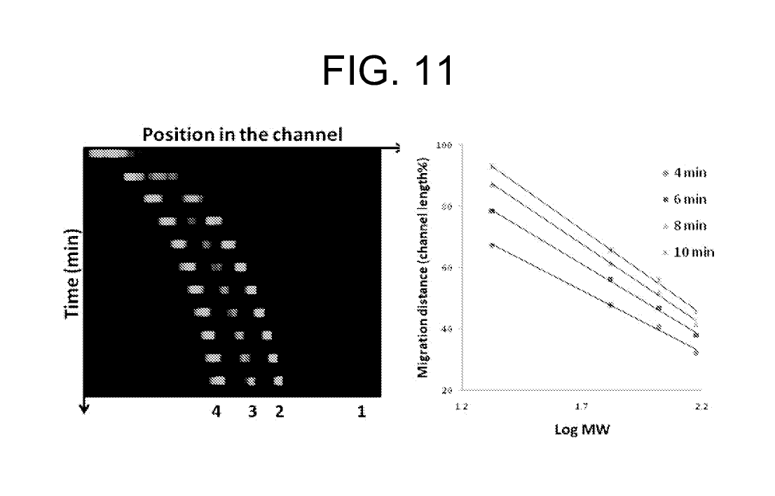

A protein ladder including Alexa Fluor 488 labeled trypsin inhibitor (TI, 21 KD), bovine serum albumin (BSA, 66 KD), C reactive protein (CRP, 115 KD) and IgG (150 kD) was injected into the PLE channel. Migration of the protein bands in the channel was shown in FIG. 11 (left panel). The velocity of the bands decreased as they migrating towards the high percentage end. Log molecular weight v.s. migration distance was plotted (right panel). Linear relationship between Log MW and migration distance was achieved.

2. PLE-IA of PSA and S100

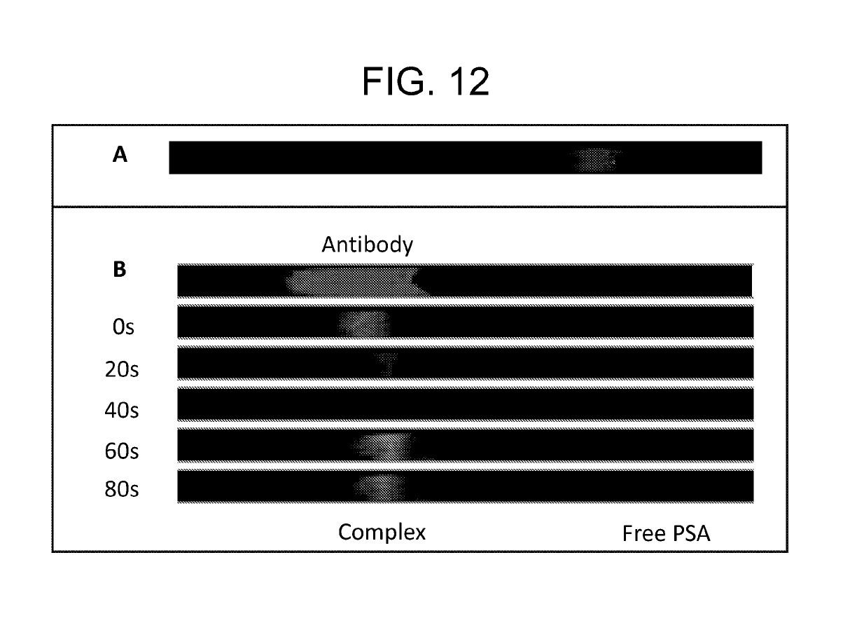

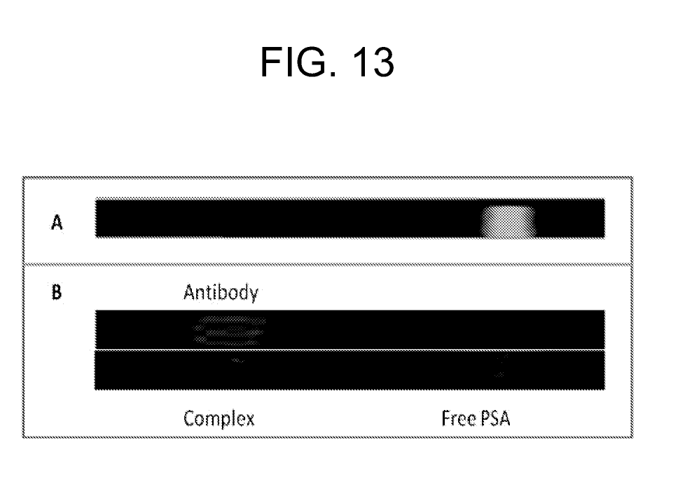

PLE-IA of PSA was demonstrated as shown in FIG. 12. A single green band was observed when Alexa Fluor 488 labeled PSA was transported to a channel without antibody. A second green band was formed which overlapped with the red antibody band when PSA was transported to a channel preloaded with Alexa Fluor 568 labeled PSA antibody, indicating the formation of PSA-PSA antibody complex. Similar to PSA, S100 PLE-IA was also demonstrated as shown in FIG. 13. One green band was observed when S100 migrated through a PLE channel without antibody while 2 bands were seen when S100 ran through a channel preloaded with S100 antibody.

3. Specificity

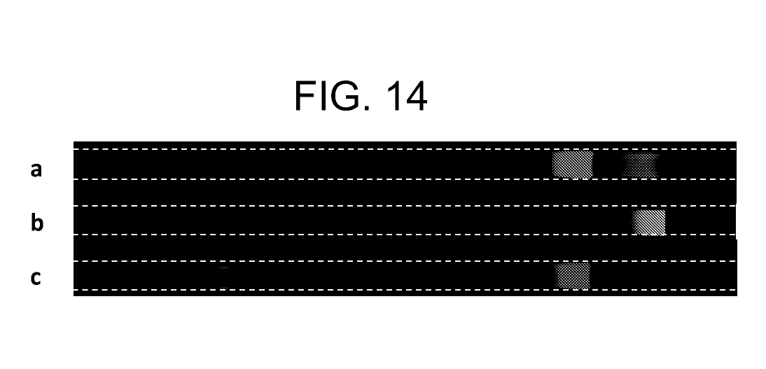

Specificity of PLE-IA was demonstrated in FIG. 14. A mixture of PSA (red) and S100 (green) was migrated through PLE channels with no antibody, with PSA antibody or with S100 antibody (antibodies were not labeled). In the channel without antibody, one green band and one red band was observed. In the channel with PSA antibody, 2 red bands were seen, indicating formation of PSA-PSA antibody complex. There was no specific binding between S100 and PSA antibody since only one green band was observed. In the channel with S100 antibody, 2 green bands and 1 red band were observed, indicating that the binding of S100 with S100 antibody was also specific. There was no detectable binding between PSA and S100 antibody.

4. Quantitative PLE-IA of Follistatin

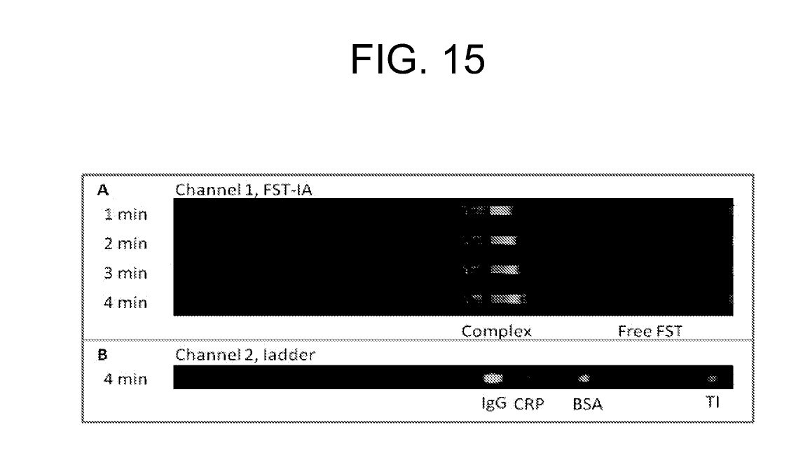

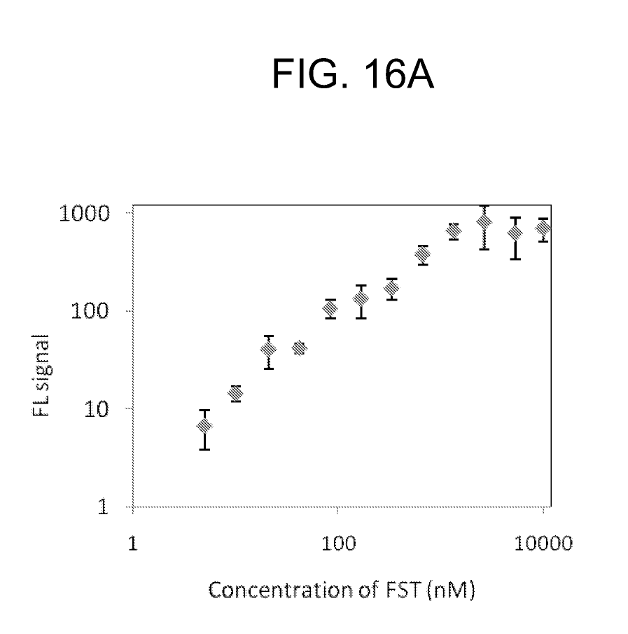

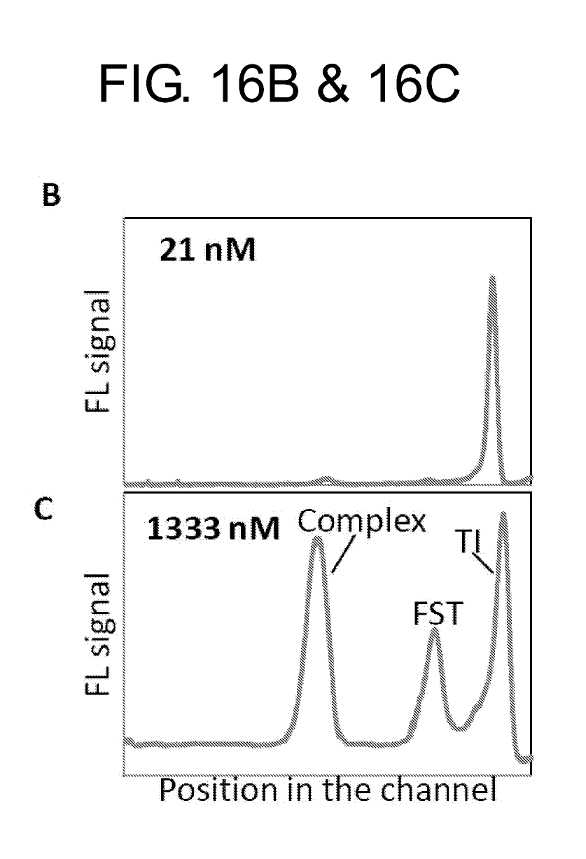

Follistatin PLE-IA was also demonstrated as shown in FIG. 15. In channel 1, follistatin (FST) was transported through preloaded FST antibody. Following incubation, free FST was separated from the FST-antibody complex as shown in panel A. In channel 2 (panel B), protein ladder were ran parallel as FST. The unbound FST was located between the TI band and BSA band, consistent with its molecular weight which is bigger than TI but smaller than BSA. FIG. 16 is the dose response curve of FST PLE-IA. FST was injected to the channel for 30 seconds. The FST plug was then transported through the antibody band under the electrical field of 50 V/cm. A linear relationship was achieved in the range from 5 nM to 1333 nM. The signal to noise ratio at 5 nM was 2.87±0.82.

5. Increased Sensitivity with Continuous Injection

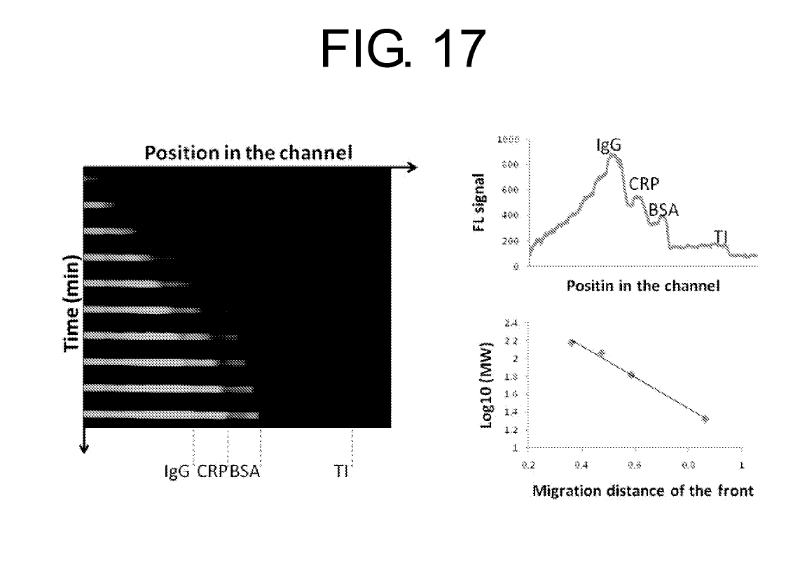

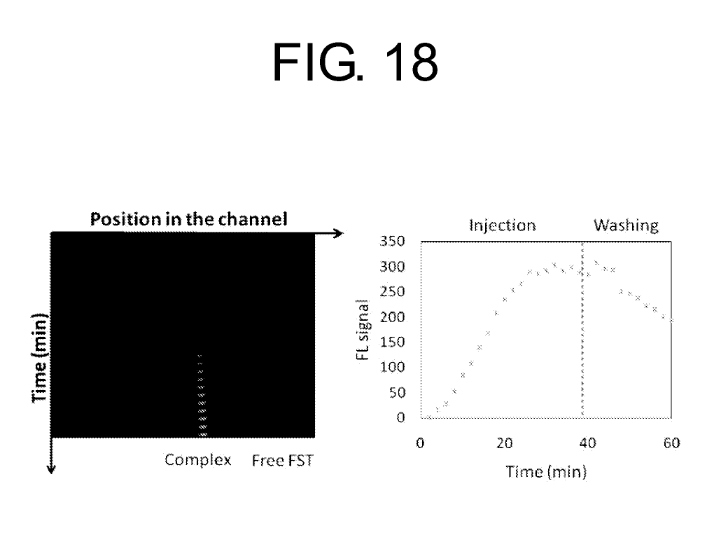

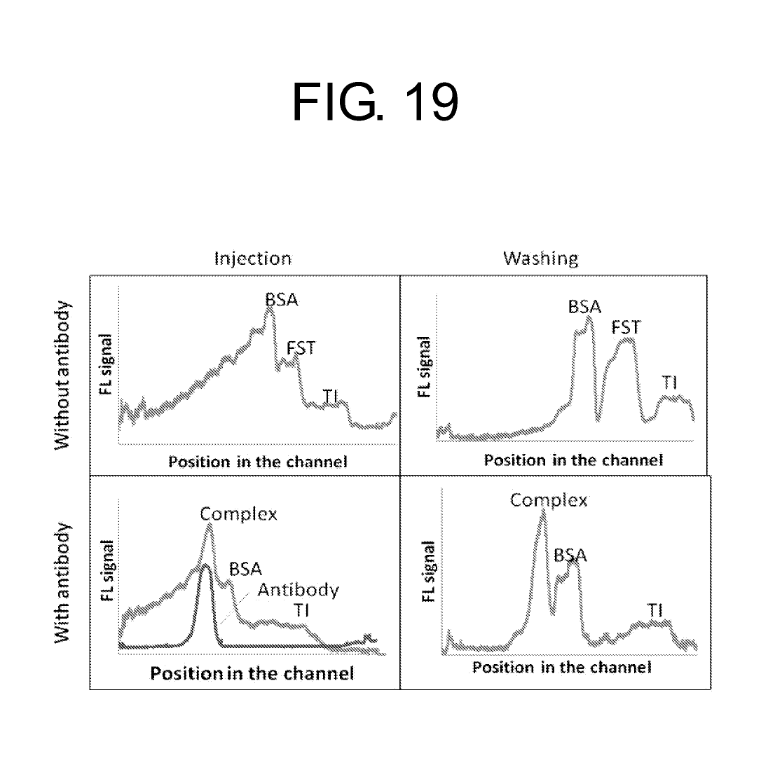

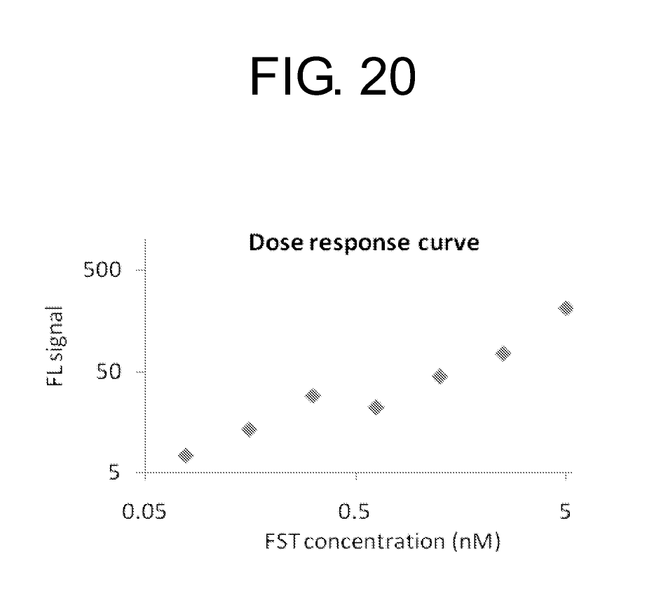

The sensitivity of FST PLE-IA increased from 5 nM to 0.078 nM when FST was injected to the channel continuously for 40 minutes as shown in FIG. 20 and Table 2. Under continuous injection, FST reached the antibody band during injection. Formation of complex continued as FST transported to the antibody band until the reaction reached equilibrium. As shown in FIG. 18 (right panel), FL signal from the complex keep increasing during injection until the reaction reached equilibrium. Then the signal dropped in the washing step when free FST passed through the antibody band and kept decreasing as bound FST dissociating from the antibody. Interestingly, information on protein size can still be obtained under continuous injection. FIG. 17 shows continuous injection of the ladder protein. Obvious front of each protein can be observed due to the concentrating effect of PLE (left panel). Similar to discontinuous injection, there is a linear relationship between log MW and the migration distance of the front of each protein band (right panel). In FIG. 19, a mixture of BSA, FST and TI was injected into PLE channels with (lower panels) or without (upper panels) antibody. The free FST peak was reduced significantly while a complex peak was formed when the mixture migrated through the channel with FST antibody.

TABLE 2
Comparison of discontinuous (30 seconds)
and continuous (40 minutes) injection

| Time of injection | 30 s | 40 min |
|---|---|---|
| Limit of Detection | 5 nM | 0.078 nM |
| SNR at 5 nM | 2.87 | 123.7 |

D. Discussion

A two-step PLE-IA was established in a microfluidic channel. Quantitative detection of target protein from prelabeled samples and readout on molecular weight was demonstrated. A few methods can be utilized to detect unlabeled samples. Fab fragment, aptamers, lectin, receptors or other molecules that bind specifically to the target protein can be used to detect the presence of the immune complex following reaction of the unlabeled protein with preloaded antibody. Alternatively, fluorescent dye that labels protein nonspecifically can be used to label all the protein bands in the PLE channel. The band of unbound target protein will be reduced when the sample migrating through preloaded antibody (FIG. 19). Information on molecular weight and concentration of the target protein can be obtained by comparing protein bands in two channels with or without preloaded antibody. Multiplexing can be accomplished by using either different sized binding partners (i.e., whole antibody, FAb, or other fragments) or by conjugating a finite size ‘shift tag’ to the pseudo-immobilized binders to shift the position of the binding partner from others, in the PLE gradient.

The microfluidic PLE provided a platform for quantitative study of immunoassays as well as other binding assays. The formation of complex can be monitored in a real time manner. Thus parameters of the binding kinetics can be measured. PLE-IA has the advantages of reduced sample volume and improved sensitivity compared to traditional methods for measuring binding kinetics such as SPR. PLE-IA provides a simple, low cost analytical platform that may have application in broad variety of areas, such as clinical diagnosis, drug screening and life science research.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method comprising:
introducing a first sample at least suspected of including a first assay member into a pore limit electrophoresis (PLE) microchannel in a manner sufficient for the first assay reagent to be pseudo-immobilized in the PLE microchannel if it is present in the sample;
introducing into the PLE microchannel a second sample at least suspected of including a second assay member that interacts with the first assay member; and
evaluating the PLE microchannel for interaction between the first and second assay reagent.

2. The method according to claim 1, wherein the first sample is known to include the first assay member.

3. The method according to claim 1, wherein the second sample is known to include the second assay member.

4. The method according to claim 1, wherein the first sample is a complex sample.

5. The method according to claim 1, wherein the second sample is a complex sample.

6. The method according to claim 1, wherein first assay member is an analyte.

7. The method according to claim 6, wherein the method is a method of determining the presence of the analyte in the sample.

8. The method according to claim 1, wherein the first assay member is an enzyme and the second assay member is a substrate for the enzyme.

9. The method according to claim 1, wherein the first assay member is an antibody and the second assay member is a specific binding member for the antibody.

10. A method of determining whether an enzyme is present in a sample, the method comprising:
separating the sample in a pore limit electrophoresis (PLE) microchannel;
introducing a substrate for the enzyme into the PLE microchannel; and
evaluating the PLE microchannel for enzyme mediated production of product from the substrate to determine whether the enzyme is present in the sample.

11. The method according to claim 10, wherein the separating comprises applying an electric field to the PLE microchannel.

12. The method according to claim 11, wherein the method further comprises determining a parameter of the enzyme when the enzyme is determined to be present in the sample.

13. The method according to claim 12, wherein the parameter is molecular weight.

14. The method according to claim 12, wherein the evaluating comprises obtaining a real-time signal from the microchannel.

15. The method according to claim 14, wherein the parameter is a kinetic parameter.

16. The method according to claim 15, wherein the kinetic parameter is kcat.

17. The method according to claim 15, wherein the kinetic parameter is Km.

18. The method according to claim 10, wherein the method has a detection sensitivity of 5 zmol or less.

19. The method according to claim 10, wherein the method comprises quantitatively determining whether the enzyme is present in the sample.

20. The method according to claim 10, wherein the method is performed in 60 min or less.

21. The method according to claim 10, wherein the sample comprises a labeled molecular weight ladder and the method further comprises detecting the ladder in the PLE microchannel.

22. The method according to claim 10, wherein the method is a method of determining the presence of two or more distinct enzymes in the sample.

23. A method of determining whether an analyte is present in a sample, the method comprising:
pseudo-immobilizing an antibody that specifically binds to the analyte in a pore limit electrophoresis (PLE) microchannel;
introducing the sample into the PLE microchannel; and
evaluating the PLE microchannel for the presence of antibody-analyte complex to determine whether the analyte is present in the sample.

24.-33. (canceled)

34. A microfluidic device comprising:
a pore-limit electrophoresis (PLE) microchannel; and
at least one of:
(a) a molecular weight ladder; and
(b) an assay reagent.

35.-50. (canceled)
